(19) United States
(12) Patent Application Publication
(10) Pub. No.: US 2011/0123988 A1
(43) Pub. Date: May 26, 2011

(54) ANTISENSE COMPOUNDS AND METHODS FOR DIAGNOSTIC IMAGING

(75) Inventors: Eric Wickstrom, Philadelphia, PA (US); Mathew L. Thakur, Cherry Hill, NJ (US)

(73) Assignee: GENESEEN LLC, CHERRY HILL, NJ (US)

(21) Appl. No.: 12/778,376

(22) Filed: May 12, 2010

Related U.S. Application Data
(63) Continuation of application No. 10/688,821, filed on Oct. 16, 2003, now abandoned.

(51) Int. Cl. C12Q 1/68; C07K 9/00

(52) U.S. Cl. 435/6; 530/322

(57) ABSTRACT

Compounds comprising a diagnostic or therapeutic moiety can be retained inside a cell by conjugating the moiety to at least one PNA that is targeted to the transcripts from a gene of interest. The diagnostic or therapeutic moiety is also conjugated to at least one targeting moiety specific for an extracellular receptor or other cell surface molecule. The targeting moiety binds to the surface of a cell, and the entire compound is then internalized. Once inside the cell, the PNA portion of the diagnostic or therapeutic compound binds to RNA transcripts in a sequence specific manner. Binding of the PNA to its target RNA transcript retains the compound within the cell. The PNA can be designed to bind to a predetermined nucleic acid sequence from an RNA transcript, for example a mutated or overexpressed sequence that is characteristic of a pathological state.

### REFERENCE TO GOVERNMENT GRANT

The invention described herein was supported in part by NIH contract no. N01-CO-27175-01. The U.S. government may have certain rights in this invention.

### FIELD OF INVENTION

This invention relates to the field of diagnostic imaging and therapy, in particular with polymeric diagnostic or therapeutic compounds conjugated to a peptide nucleic acid.

### BACKGROUND OF THE INVENTION

To date, a total of about two hundred different genes are believed to be mutated in the various known cancers. A gene associated with a cancer can also carry multiple mutations. The particular combination of intra- or inter-genic mutations is therefore unique to a given cancer, and can even vary between individuals with the same cancer type.

For example, diffuse large B-cell lymphoma (DLBCL) can be classified into two subgroups with significantly different survival rates, based on gene expression profiles. A number of mutations have also been identified in the 12^{th} codon of the K-Ras oncogene, where the different mutations can lead to colon, lung, or pancreatic cancer. Various mutations in the tumor suppressor gene p53 have also been characterized in some fraction of all tumor types, particularly in pancreatic ductal carcinomas. Over-expression of oncogenes such as CCND1, HER2, and MYC, has been associated with various types of cancer, in particular pancreatic and breast cancers. Over-expression of these oncogenes can occur through mutations in the regulatory sequences of these genes, by gene amplification, or by gene rearrangement.

The classification of clinical symptoms in a cancer patient, without characterizing the underlying mutations in the cancer cells, does not provide the information needed to effectively treat the disease in that patient. Furthermore, the underlying mutations or the overexpression of oncogenes can be detected prior to the presentation of clinical symptoms, allowing treatment to begin at a much earlier stage and improving patient prognosis. This is particularly important for aggressive cancers, such as pancreatic cancer, in which the patient does not present with symptoms until the disease is well advanced. For example, the current median survival time for pancreatic cancer patients is 12 months, with only 1% of patients surviving more than 5 years after diagnosis. Early detection of oncogene over-expression in patients at risk for pancreatic cancer could significantly increase their mean survival time.

Current diagnostic imaging modalities are unable to identify or measure levels of specific mRNAs in vivo. Anatomic imaging by computed tomography or magnetic resonance imaging can provide structural details of tumors, but provides no information on the type or level of oncogene expression in cancer cells. Combinations of anatomic imaging with metabolic position emission tomography (PET) yield variable results. Fluorescence and luminescence imaging show promise for functional imaging of tumors, but are severely limited in depth of tissue penetration and would be of little use in imaging the viscera. Moreover, many of the constructs currently used for preclinical imaging, such as those containing luciferase, are toxic in humans.

The development of PET imaging with ¹⁸F-Fluorodeoxy-glucose or ¹⁸F-fluoroguanine derivatives may allow the physician to identify sites of cellular proliferation in vivo. However, such imaging techniques identify genes that are mutated or overexpressed in the proliferating cells.

Gene expression profiling has been used to classify cutaneous malignant melanoma, breast cancer tumors, and to identify genes important for metastasis. Such profiling studies allow the segregation of distinct groups from an otherwise indistinguishable patient population. Nevertheless, the expression profiles have not provided clear directions for developing an effective molecular therapy.

Even if the sequence of the mutated gene is known, it is often difficult to concentrate enough imaging or diagnostic agent in a cell for visualization or therapy of a cell. Polymeric or dendrimeric “magnifiers” have been developed, which carry numerous imaging or therapeutic moieties per molecule. For example, a large “starburst” dendrimer can deliver 256 gadolinium ions (for magnetic resonance imaging) or rhenium radionuclides (for therapeutic uses) into a given cell. However, multiple polymeric or dendrimeric compounds are still required for effective imaging or therapy of cells containing a mutated gene. It is therefore desirable to target the transcript (e.g., mRNA) of a gene of interest, as many thousands of transcript molecules can be present inside the cell. If the transcripts from a given mutated gene can be specifically targeted, a sufficient number of diagnostic or therapeutic compounds could be retained inside a cell for effective imaging or treatment of that cell.

In some cases, the molecular targeting of transcripts from a known oncogene has produced encouraging therapeutic results. For example, targeting the BCR/ABL product with the drug STI-571 has shown some promise in treating chronic myelogenous leukemia, although resistance to the drug develops quickly.

Inhibition of BCL-2 expression with traditional antisense agents showed a clinical response in follicular lymphoma, melanoma and prostate cancer. However, such antisense agents can have problems relating to toxicity, stability and efficacy.

Nucleic acid analogs have also been developed for use as anti-oncogenic antisense agents. These analogs have modifications that improve biological stability, solubility, cellular uptake and ease of synthesis. The simplest modification involves blocking the 3′ terminus of the nucleic acid to prevent exonucleolytic degradation. Other modifications focus on preserving inter-nucleoside linkages by changing the normal phosphodiester bonds to phosphorothioates, methylphosphonates or boranophosphonates. However, such modifications weaken hybridization of the antisense agent to the target mRNA. Phosphorothioate-modified antisense agents can also be toxic.

The antisense agents discussed above are also not useful as diagnostic agents, because the molecules are negatively charged. Hybridization of these charged antisense nucleic acids to a target mRNA forms a substrate for RNase H. The gene transcript that one wishes to detect is therefore destroyed upon administration of the antisense agent to the cell. Moreover, most nucleic acids or nucleic acid analogs are taken up non-specifically by cells, and it is often difficult to prevent a general distribution of antisense agents into cells of the body.

Peptide nucleic acids (PNA) are uncharged nucleic acid analogs in which the phosphodiester linkages and sugar moieties are replaced with a peptide-like backbone of (N-2-aminoethyl) glycine units. The purine and pyrimidine bases are attached to the peptide-like backbone by methylene-carbonyl linkers. Compared with other nucleic acid analogs, PNAs have the highest Tₘs for duplexes formed with single-stranded DNA or RNA. The PNAs can also be made nuclease resistant without loss of basepairing efficiency by reversing the attachment of the base to the backbone, thus changing the natural β-anomer to the α-anomer.

PNAs are also not generally taken up by cells; introduction of PNAs into cells is accomplished by techniques such as microinjection or by conjugating the PNA to a cell targeting moiety. Non-specific distribution of PNA compounds into non-target cells can therefore be avoided.

Insulin like growth factor 1 (IGF1) and its receptor IGFR1 play a major regulatory role in the development, cell cycle progression, and early phase of tumorigenicity in most cancerous cells. For example, the IGF1 receptor gene is amplified in about 70% of human pancreatic tumors, and has been exploited as an antisense target in brain cancer. IGF1 peptide analogs act as IGFR1 antagonists inhibit the growth of certain cancer cell lives. Other cell surface markers are also known to be specific to cancer cells.

What is needed, therefore, are methods and compounds that allow the non-invasive and effective detection of gene expression in vivo in a chosen cell, where the gene expression is detected with high specificity and sensitivity. The compounds should be stable, non-toxic, and should not cause degradation of mRNA expressed from the gene of interest. Ideally, the compounds used to detect gene expression can also be used therapeutically in the same cells by substituting a therapeutic moiety for a detectable moiety; for example by substituting rhenium radionuclides for gadolinium ions in the compound.

### SUMMARY OF THE INVENTION

It has now been discovered that a compound comprising a diagnostic or therapeutic moiety can be retained inside a cell by conjugating the moiety to at least one PNA that is targeted to the transcripts from one or more genes of interest. The diagnostic or therapeutic moiety is also conjugated to at least one targeting moiety specific for an extracellular receptor or other cell surface molecule. The targeting moiety binds to the surface of a cell, and the entire compound is then internalized. Once inside the cell, the PNA portion of the diagnostic or therapeutic compound binds to RNA transcripts in a sequence specific manner. Binding of the PNA to its target RNA transcript retains the compound within the cell. The PNA can be designed to bind to a predetermined nucleic acid sequence from an RNA transcript, for example a mutated or overexpressed sequence that is characteristic of a pathological state. In a preferred embodiment, the diagnostic or therapeutic moiety is a polymeric diagnostic or therapeutic moiety.

The invention thus provides a compound comprising a diagnostic or therapeutic moiety conjugated to a PNA and a targeting moiety, wherein the PNA comprises a base sequence that is complementary to a target nucleic acid sequence within a cell.

In one embodiment, the compound comprises the formula

X-L₁-Y

wherein:

X is a diagnostic or therapeutic moiety;

L₁ is a chemical bond or at least one linking moiety; and

Y is P-L₂-T or T-L₂-P, in which
P is at least one peptide nucleic acid comprising a base sequence that is complementary to the target nucleic acid sequence;
L₂ is a chemical bond or at least one linking moiety; and
T is at least one targeting moiety.

The invention also provides a diagnostic imaging method, comprising contacting cells of a subject with a compound comprising a diagnostic moiety conjugated to at least one PNA and at least one targeting moiety. The cells contain transcripts comprising a target nucleic acid sequence indicative of a pathological state, and the PNA comprises a base sequence that is complementary to a target nucleic acid sequence within a cell. The compound binds to the cell via the targeting moiety and is internalized by the cell, whereupon the PNA binds to the target nucleic acid sequence and retains the compound inside the cell. The compound can then be detected within the cell, wherein the presence of the compound within the cell indicates a pathological state.

The invention further provides a therapeutic method, comprising contacting cells of a subject with a compound comprising a therapeutic moiety conjugated to at least one PNA and at least one targeting moiety. The cells contain transcripts comprising a target nucleic acid sequence indicative of a pathological state, and the PNA comprises a base sequence that is complementary to a target nucleic acid sequence within a cell. The compound binds to the cell via the targeting moiety and is internalized by the cell, whereupon the PNA binds to the target nucleic acid sequence and retains the compound inside the cell. The presence of the compound within the cell inhibits growth of the cell, or causes death of the cell.

The invention also provides a method of retaining a compound inside a cell, comprising contacting a cell that contains transcripts comprising a target nucleic acid sequence with a compound comprising a diagnostic or therapeutic moiety conjugated to at least one PNA and at least one targeting moiety. The PNA comprises a base sequence that is complementary to a target nucleic acid sequence within a cell. The compound binds to the cell via the targeting moiety and is internalized by the cell, whereupon the PNA binds to the target nucleic acid sequence and retains the compound inside the cell.

The invention still further provides a method for detecting the overexpression of a transcript comprising a target nucleic acid sequence within a cell, comprising contacting a cell suspected of overexpressing the transcript with a compound comprising a diagnostic moiety conjugated to at least one PNA and at least one targeting moiety. The PNA comprises a base sequence that is complementary to a target nucleic acid sequence within a cell. The compound binds to the cell via the targeting moiety and is internalized by the cell, whereupon the PNA binds to the target nucleic acid sequence and retains the compound inside the cell. The compound can then be detected within the cell, wherein the presence of the compound within the cell indicates over-expression of the nucleic acid.

### DETAILED DESCRIPTION OF THE INVENTION

The invention is a compound comprising a diagnostic or therapeutic moiety conjugated to at least one PNA and at least one targeting moiety, that is able to penetrate a given cell and selectively bind to RNA transcripts within that cell. The PNA comprises a base sequence that is complementary to a target nucleic acid sequence within a cell. The targeting moiety comprises a molecule that binds to, or is bound by, a cell surface molecule on a cell of interest (for example a tumor cell).

The diagnostic or therapeutic moiety can comprise a molecule which carries a single diagnostic or therapeutic center into a cell. As used herein, a “diagnostic center” comprises an atom or molecule that can be detected, such as an ultrasound agent, a fluorescent molecule, a paramagnetic metal ion, a heavy metal ion or an ion of a radioactive isotope. Preferably, the diagnostic center comprises a chelated metal ion. As used herein, a “therapeutic center” is an atom or molecule that slows or halts the growth of a cell, or causes the death of a cell. For example, a therapeutic center can be a chemical or radioactive moiety that damages vital cellular structures or interrupts vital cell processes. Preferably, the therapeutic center comprises a chelated ion of a radioactive metal isotope. Diagnostic and therapeutic centers are described in more detail below. The compounds of the invention can therefore be used for imaging or killing of cells containing specific RNA transcripts. In particular, the present compounds can image or kill cells overexpressing an oncogene.

In a preferred embodiment, the diagnostic or therapeutic moiety comprises a polymeric diagnostic or therapeutic agent. Surprisingly, the relatively large polymeric diagnostic or therapeutic moiety does not prevent the PNA portion of the present compounds from binding to the target RNA. As used herein, a “polymeric diagnostic or therapeutic moiety is designed to carry a plurality of diagnostic or therapeutic centers into a cell.

The polymeric diagnostic or therapeutic moieties of the invention can comprise a linear or branched polymer, for example a linear or branched oligomeric polychelant comprising alternating chelant and linker moieties bound together by amide or ester moieties, as described in U.S. Pat. No. 5,446,145, the entire disclosure of which is herein incorporated by reference. Other linear polymeric chelant moieties are known in the art, for example those in which chelant groups are pendant from a polylysine, polyamine or polyalkylene oxide backbone. The diagnostic or therapeutic moiety can also comprise a branched polymer or a “dendrimer,” as described by Tomalia et al., Polymer Journal 17: 117, 1985 and in U.S. Pat. No. 4,587,329, the entire disclosures of which are herein incorporated by reference. Preferred polymeric diagnostic or therapeutic moieties comprise dendrimers.

Dendrimers are polymers with densely branched structures having a large number of reactive groups. A dendrimer includes several layers or generations of repeating units that all contain one or more branch points. As used herein, a dendrimer includes generally any of the known dendritic architectures, including starburst dendrimers, cascade dendrimers and controlled and random hyperbranched dendrimers, as described in U.S. Pat. No. 6,475,994, the entire disclosure of which is herein incorporated by reference. Dendrimers that are particularly well suited for use in the present compounds include those containing exterior and/or interior primary or secondary amine groups, amide groups, or combinations thereof. Such dendrimers include polyamidoamine (PAMAM) dendrimers, polypropylamine (POPAM) dendrimers, polyether (PE) and polyethyleneimine (PEI) dendrimers.

Dendrimers are generally prepared by condensation reactions of monomeric units having at least two reactive groups, for example by convergent or divergent synthesis. Divergent synthesis of dendrimers involves a molecular growth process that occurs through a consecutive series of geometrically progressive additions of branched molecule upon branched molecule in a radially outward direction, to produce an ordered arrangement of layered branches. Convergent synthesis of dendrimers involves a growth process that begins from what will become the surface of the dendrimer, which progresses radially in a direction toward the dendrimer focal point or core. Preferably, dendrimers are synthesized by divergent synthesis.

Each dendrimer includes a core molecule or “core dendron,” one or more layers of internal dendrons, and an outer layer of surface dendrons, wherein each of the dendrons includes a single branch juncture. As used herein, “dendrons” are branched molecules that are used to construct a dendrimer generation. The dendrons can be the same or different in chemical structure and branching functionality. The branches of dendrons can contain either chemically reactive or passive functional groups. Chemically reactive surface groups can be used for further extension of dendritic growth or for modification of dendritic molecular surfaces, for example by attachment of targeting moieties or PNAs. The chemically passive groups can be used to physically modify dendritic surfaces, such as to adjust the ratio of hydrophobic to hydrophilic terminals, and/or to improve the solubility of the dendrimer for a particular environment.

Dendrimers can be described by reference to their “generation,” or the number of synthetic rounds that the dendrimer has undergone. For example, the starting or “core” dendron is generation zero. The first addition of dendrons onto the core dendron is the first generation. The second addition of dendrons onto the core dendron is the second generation, and so on. Reference to the generation can provide information about the number of endgroups available for conjugation with other moieties, for example with diagnostic or therapeutic centers. Thus, a PAMAM starburst dendrimer with four amines on the core dendron at generation zero will have eight amines after the first generation, sixteen amines after the second generation, 32 amines after the third generation, and so forth. Preferred starburst dendrimers are those of the sixth generation starting from a core dendron having four reactive groups, to give a dendrimer with 256 reactive groups.

Hyperbranched dendrimers are dendrimers that contain high levels of irregular branching, as compared with the more nearly perfect regular structure of starburst or cascade dendrimers. Specifically, hyperbranched dendrimers contain a relatively high number of irregular branching areas, in which not every repeat unit contains a branch juncture. The preparation and characterization of random and controlled hyperbranched polymers is within the skill in the art, for example as described in U.S. Pat. Nos. 4,631,337; 4,694,064; 4,713,975; 4,737,550; 4,871,779 and 4,857,599 and 5,418,301, the entire disclosures of which are herein incorporated by reference.

Particularly preferred dendrimers for use in the invention include the dense star polymers or starburst polymers, for example as described in U.S. Pat. Nos. 4,507,466, 4,558,120, 4,568,737 and 4,587,329, the entire disclosures of which are herein incorporated by reference. In addition to their ability to carry multiple diagnostic or therapeutic centers conjugated to surface reactive groups, starburst dendrimers also exhibit “starburst dense packing” at high generations. “Starburst dense packing” refers to the situation where the surface of the dendrimer contains sufficient terminal moieties such that the dendrimer surface becomes congested and encloses void spaces within the interior of the dendrimer. This congestion can provide a molecular barrier that can be used to entrap diagnostic or therapeutic centers for delivery into a cell.

Preparation of Starburst Dendrimers for Use in the Invention is within the Skill in the art; e.g., as described in U.S. Pat. No. 4,587,329, supra. For example, polyamine starburst dendrimers can be prepared by reacting ammonia or an amine having a plurality of primary amine groups with N-substituted aziridine, such as N-tosyl or N-mesyl aziridine, to form a protected first generation polysulfonamide. The first generation polysulfonamide is then activated with acid, such as sulfuric, hydrochloric, trifluoroacetic, fluorosulfonic or chlorosulfonic acid, to form the first generation polyamine salt. Preferably, the desulfonylation is carried out using a strong acid that is volatile enough to allow removal by distillation, such as hydrochloric acid. The first generation polyamine salt can then be reacted further with N-protected aziridine to form the protected second generation polysulfonamide. The sequence can be repeated to produce higher generation polyamine dendrimers.

Polyamidoamine starburst dendrimers can be prepared by first reacting ammonia with methyl acrylate under conditions sufficient to cause the Michael addition of one molecule of the ammonia to three molecules of the methyl acrylate to form the core adduct. Following removal of unreacted methylacrylate, the core adduct is reacted with excess ethylenediamine, under conditions such that one amine group of the ethylenediamine molecule reacts with the methyl carboxylate groups of the core adduct to form a first generation adduct having three amidoamine moieties. Following removal of unreacted ethylenediamine, this first generation adduct is then reacted with excess methyl acrylate under Michael addition conditions to form a second generation adduct having terminal methyl ester moieties. The second generation adduct is then reacted with excess ethylenediamine under amide forming conditions to produce the desired polyamidoamine dendrimer having ordered, second generation dendritic branches with terminal amine moieties. Similar dendrimers containing amidoamine moieties can be made by using organic amines as the core compound; e.g., ethylenediamine, which produces a tetra-branched dendrimer or diethylenetriamine that produces a penta-branched dendrimer.

The surface chemistry of the dendrimers can be controlled in a predetermined fashion by selecting a repeating unit that contains the desired chemical functionality or by chemically modifying all or a portion of the surface functionalities to create new surface functionalities. These surfaces functionalities can then be used to conjugate diagnostic or therapeutic centers, targeting moieties or PNAs to the surface of the dendrimer.

For example, the dendrimers for use in the present compounds can have terminal groups that are sufficiently reactive to undergo addition or substitution reactions. Examples of such terminal groups include amino, hydroxy, mercapto, carboxy, alkenyl, allyl, vinyl, amido, halo, urea, oxiranyl, aziridinyl, oxazolinyl, imidazolinyl, sulfonato, phosphonato, isocyanato and isothiocyanato. The terminal groups can also be modified to make the dendrimers biologically inert, for example, to make the dendrimers non-immunogenic or to avoid non-specific uptake of the dendrimer by the liver, spleen or other organ. Techniques for modifying the terminal groups of dendrimers are within the skill in the art, for example as described in U.S. Pat. No. 6,177,414, the entire disclosure of which is herein incorporated by reference.

The diagnostic or therapeutic moiety can also comprise dendrimers or other polymers with at least one biodegradation cleavage site, as described in U.S. Pat. No. 5,834,020, the entire disclosure of which is herein incorporated by reference. On cleavage of the dendrimers or other polymers at the biodegradation cleavage site, diagnostic or therapeutic centers and fragments of the backbone are released in renally excretable form. Thus, compounds of the invention that are not taken up by cells can be readily cleared from the blood stream. The diagnostic or therapeutic moiety can also comprise two or more dendrimers and/or other polymers conjugated together to create “bridged” dendrimeric or polymeric moieties.

In one embodiment, a diagnostic moiety of the invention comprises a compound conjugated to a single diagnostic center. In a preferred embodiment, a diagnostic moiety of the invention is formed by conjugating a polymer, preferably a dendrimer, with a plurality of diagnostic centers. As used herein, “conjugated” means that two chemical moieties are joined by a chemical bond or by a linking moiety. Examples of chemical bonds are covalent, hydrophilic, ionic, or hydrogen bonds. A preferred chemical bond is a covalent bond.

A preferred diagnostic center comprises a diagnostic metal ion or a non-metal radioisotope (e.g. a radioactive halogen). As used herein, a “diagnostic metal ion” is a paramagnetic metal ion (e.g., of atomic number 21 to 29, 42, 44 and 57 to 71, especially 24 to 29 and 62 to 69), a heavy metal ion (e.g., of atomic number 37 or more preferably 50 or more) or an ion of a radioactive metal isotope. Preferred paramagnetic metal ions are Eu, Ho, Gd, Dy, Mn, Cr and Fe, and particularly preferred paramagnetic ions are Gd(III), Mn(II) and Dy(III). Preferred heavy metal ions are Hf, La, Yb, Dy and Gd. Preferred radioactive isotopes are useful for scintigraphy, SPECT or PET imaging. For use in PET imaging, one of the various positron emitting metal ions, such as ⁵¹Mn, ⁵²Fe, ⁶⁰Cu, ⁶⁸Ga, ⁷²As, ^{94m}Tc, or ¹¹⁰In is preferred. Preferred isotopes for labeling by halogenation include ¹⁸F, ¹²⁴I, ¹²⁵I, ¹³¹I, ¹²³I, ⁷⁷Br, and ⁷⁶Br. Preferred radioactive metal isotopes for scintigraphy include ⁶⁴Cu, ⁶⁷Ga, ⁶⁸Ga, ⁸⁷Y, ^{99m}Tc, and ¹¹¹In. ^{99m}Tc is particularly preferred for diagnostic applications because of its low cost, availability, imaging properties, and high specific activity. The nuclear and radioactive properties of Tc-99m make this isotope an ideal scintigraphic imaging agent. This isotope has a single photon energy of 140 keV and a radioactive half-life of about 6 hours, and is readily available from a ⁹⁹Mo-^{99m}Tc generator.

In one embodiment the diagnostic center is a chelant able to complex a diagnostic metal ion. For use as a diagnostic moiety, the diagnostic center is complexed with the diagnostic metal ion. Suitable chelants (or chelators) for complexing diagnostic metal ions include NxSy chelants that have cores of the following configurations: N2S2 (e.g., as described in U.S. Pat. No. 4,897,225; U.S. Pat. No. 5,164,176; or U.S. Pat. No. 5,120,526); N3 (e.g., as described in U.S. Pat. No. 4,965,392); N2S3 (e.g., as described in U.S. Pat. No. 4,988,496), N2S4 (e.g., as described in U.S. Pat. No. 4,988,496), N3S3 (e.g., as described in U.S. Pat. No. 5,075,099); N4 (e.g., as described in U.S. Pat. No. 4,963,688 and U.S. Pat. Nos. 5,227,474, 6,143,274, 6,093,382, 5,608,110, 5,665,329, 5,656,254 and 5,688,487) or NS3. Certain N₃S chelants are described in PCT/CA94/00395, PCT/CA94/00479, PCT/CA95/00249 and in U.S. Pat. Nos. 5,662,885; 5,976,495; and 5,780,006. The chelator may also include derivatives of the chelating ligand mercapto-acetyl-acetyl-glycyl-glycine (MAG3), which contains an N₃S, and N₂S₂ systems such as MAMA (monoamidemonoaminedithiols), DADS (N₂S diaminedithiols), CODADS and the like. These chelator systems and a variety of others are described in Liu and Edwards, Chem. Rev. 1999, 99, 2235-2268 and references therein.

The chelant may also include complexes containing ligand atoms that are not donated to the metal in a tetradentate array. These include the boronic acid adducts of technetium and rhenium dioximes, such as are described in U.S. Pat. Nos. 5,183,653; 5,387,409; and 5,118,797, the disclosures of which are incorporated by reference herein, in their entirety.

Preferred NxSy chelants comprise N2S2, N3S or N4 cores. Exemplary NxSy chelants are also described in Fritzberg et al., P.N.A.S. USA 85:4024-29, 1988 and Weber et al., Bioconj. Chem. 1:431-37, 1990. The disclosures of the journal articles and U.S. patents identified in this paragraph are herein incorporated by reference in their entirety.

Methods for conjugating NxSy chelants to dendrimers and other polymers are known in the art; for example as disclosed in U.S. Pat. No. 5,175,257 and U.S. Pat. No. 6,171,577, the entire disclosures of which are herein incorporated by reference. Preferably, the NxSy chelant is conjugated to the dendrimer by a chemically reactive “linking moiety,” which is reactive under conditions that do not denature or otherwise adversely affect the chelant or polymer. The linking moiety can be separate from, or integral to, the chelant. Chelants that have integral linking moieties are known as “bifunctional chelants.”

Linking moieties may include, without limitation, amide, urea, acetal, ketal, double ester, carbonyl, carbamate, thiourea, sulfone, thioester, ester, ether, disulfide, lactone, imine, phosphoryl, or phosphodiester linkages; substituted or unsubstituted saturated or unsaturated alkyl chains; linear, branched, or cyclic amino acid chains of a single amino acid or different amino acids (e.g., extensions of the N- or C-terminus of the binding moieties); derivatized or underivatized polyethylene glycol, polyoxyethylene, or polyvinylpyridine chains; substituted or unsubstituted polyamide chains; derivatized or underivatized polyamine, polyester, polyethylenimine, polyacrylate, poly(vinyl alcohol), polyglycerol, or oligosaccharide (e.g., dextran) chains; alternating block copolymers; malonic, succinic, glutaric, adipic and pimelic acids; caproic acid; simple diamines and dialcohols; any of the other linkers disclosed herein; or any other simple polymeric linkers known in the art (see, e.g., WO 98/18497, WO 98/18496). Preferably the molecular weight of the linker can be tightly controlled. In one embodiment, the molecular weights can range in size from less than 100 to greater than 1000. Preferably the molecular weight of the linker is less than 100. In addition, it may be desirable to utilize a linker that is biodegradable in vivo to provide efficient routes of excretion for the imaging reagents of the present invention. Depending on their location within the linker, such biodegradable functionalities can include ester, double ester, amide, phosphoester, ether, acetal, and ketal functionalities. Particularly suitable linking moieties include active esters, isothiocyanates, amines, hydrazines, maleimides or other Michael-type acceptors, thiols, and activated halides. Among the preferred active esters are N-hydroxysuccinimidyl ester, sulfosuccinimidyl ester, thiophenyl ester, 2,3,5,6-tetrafluorophenyl ester, and 2,3,5,6-tetrafluorothiophenyl ester

Other suitable chelants for use in the present invention include linear, cyclic and branched polyamino-polycarboxylic acids and their phosphorous oxyacid equivalents, for example ethylenediamine-N,N,N′,N′-tetraacetic acid (EDTA); N,N,N′,N″,N″-diethylene-triaminepentaacetic acid (DTPA); 1,4,7,10-tetraazocyclododecane-N,N′N″,N″′-tetraacetic acid (DOTA); 1,4,7,10-tetraazo-cyclododecane-N,N′N″-triacetic acid (DO3A); 1-oxa-4,7,10-triazacyclododecane-N,N′N″-triacetic acid (OTTA); trans(1,2)-cyclohexanodiethylene-triamine-pentaacetic acid (CDTPA); 1-oxa-4,7,10-triazacyclododecantriaacetic acid (DOXA); 1,4,7-triazacyclononanetriacetic acid (NOTA); and 1,4,8,11-tetraazacyclotetradecanetetraacetic acid (TETA). DOTA and DO3A are preferred.

Such chelants can be linked to dendrimers or other polymers by any suitable method, e.g. as described in WO 90/12050 and WO 93/06868 and in U.S. Pat. No. 5,364,613 and U.S. Pat. No. 6,274,713, the entire disclosures of which are herein incorporated by reference. For example, the chelant can be linked to the dendrimer or other polymer via one of the metal coordinating groups, which can form an ester, amide thioester or thioamide bond with an amine, thiol or hydroxy group on the dendrimer. Alternatively, the chelant can be linked to a dendrimer via a functional group attached directly to the chelant; e.g., a CH₂-phenyl-NCS group attached to a ring carbon of DOTA as described in Meares et al., JACS 110: 6266-6267, the entire disclosure of which is herein incorporated by reference. The chelant can also be linked to a dendrimer indirectly with a homo- or hetero-bifunctional linking moiety; e.g., a bis amine, bis epoxide, diol, diacid, or a difunctionalized PEG. As above, chelants that have integral linking moieties are known as “bifunctional chelants.”

Suitable methods for metallating chelants with an imaging or therapeutic radionuclide are within the skill in the art; e.g., as described in U.S. Pat. No. 5,175,257 and U.S. Pat. No. 6,171,577, the entire disclosures of which are herein incorporated by reference. For example, imaging or therapeutic radionuclides can be incorporated into a compound of the invention by direct incorporation, template synthesis and/or transmetallation. Direct incorporation is preferred.

For direct incorporation, the imaging metal ion must be easily complexed by the chelant; for example, by merely exposing or mixing an aqueous solution of chelant-containing compound with a metal salt in an aqueous solution. The metal salt can be any salt, but is preferably a water-soluble salt of the metal such as a halogen salt. More preferably, such salts are selected so as not to interfere with the binding of the metal ion with the chelant. The chelant-containing compound can be mixed with buffer salts such as citrate, acetate, phosphate and/or borate to produce the optimum pH for the direct incorporation.

The metal ion can be complexed with the chelant at any suitable stage in the synthesis of the present diagnostic imaging compound. Preferably, the metal ion is complexed with the chelant after the chelant is conjugated to the dendrimer or other polymer, and more preferably after the PNA and targeting moieties have also been conjugated to the dendrimer or other polymer.

In another embodiment, the diagnostic center can comprise an ultrasound contrast agent. Gas containing or gas generating ultrasound contrast agents are particularly useful because of the acoustic difference between liquid (e.g., blood) and the gas-containing or gas generating ultrasound contrast agent. Because of their size, ultrasound contrast agents comprising microbubbles, microballoons, and the like may remain for a longer time in the blood stream after injection than other detectable moieties; thus a targeted ultrasound agent may demonstrate superior imaging of tissue expressing or containing the target.

In this aspect of the invention, the diagnostic center may include a material that is useful for ultrasound imaging. For example, the diagnostic center may include materials employed to form vesicles (e.g., microbubbles, microballoons, microspheres, etc.), or emulsions containing a liquid or gas which functions as the detectable label (e.g., an echogenic gas or material capable of generating an echogenic gas). Materials for the preparation of such vesicles include surfactants, lipids, sphingolipids, oligolipids, phospholipids, proteins, polypeptides, carbohydrates, and synthetic or natural polymeric materials. See e.g. WO 98/53857, WO 98/18498, WO 98/18495, WO 98/18497, WO 98/18496, and WO 98/18501 incorporated herein by reference in their entirety.

For contrast agents comprising suspensions of stabilized microbubbles (a preferred embodiment), phospholipids, and particularly saturated phospholipids are preferred. The preferred gas-filled microbubbles can be prepared by means known in the art, such as, for example, by a method described in any one of the following patents: EP 554213, U.S. Pat. No. 5,413,774, U.S. Pat. No. 5,578,292, EP 744962, EP 682530, U.S. Pat. No. 5,556,610, U.S. Pat. No. 5,846,518, U.S. Pat. No. 6,183,725, EP 474833, U.S. Pat. No. 5,271,928, U.S. Pat. No. 5,380,519, U.S. Pat. No. 5,531,980, U.S. Pat. No. 5,567,414, U.S. Pat. No. 5,658,551, U.S. Pat. No. 5,643,553, U.S. Pat. No. 5,911,972, U.S. Pat. No. 6,110,443, U.S. Pat. No. 6,136,293, EP 619743, U.S. Pat. No. 5,445,813, U.S. Pat. No. 5,597,549, U.S. Pat. No. 5,686,060, U.S. Pat. No. 6,187,288, and U.S. Pat. No. 5,908,610, each of which is incorporated by reference herein in its entirety. The agents can be conjugated to the PNA and targeting moiety directly or via one or more linking groups as known in the art and described herein.

As discussed above, a therapeutic moiety of the invention can comprise a compound conjugated to a single therapeutic center. In a preferred embodiment, a therapeutic moiety of the invention can be formed by conjugating a polymer, preferably a dendrimer, with a plurality of therapeutic centers. A preferred therapeutic center comprises a therapeutic radionuclide. In one embodiment, the therapeutic center is a chelant complexed to a therapeutic metal ion. As used herein, a “therapeutic metal ion” is an ion of a radioactive metal isotope suitable for use in radiotherapy; for example ⁶⁴Cu, ⁹⁰Y, ¹⁰⁵Rh, ¹¹¹In, ^{117m}Sn, ¹⁴⁹Pm, ¹⁵³Sm, ¹⁶¹Tb, ¹⁶⁶Dy, ¹⁶⁶Ho, ¹⁷⁵Yb, ¹⁷⁷Lu, ^{186/188}Re, ¹⁹⁹Au, ⁴⁷Sc, ⁶⁷Cu, ⁶⁷Ga, ²¹²Pb, ⁶⁸Ga, ²¹²Bi, ²¹⁰At, and ²¹¹At. Preferred therapeutic radionuclides are ⁹⁰Y, ¹⁸⁶Re and ¹⁸⁸Re. An appropriate chelant, including those described above for the diagnostic centers, can be used to complex the therapeutic metal ions. Likewise, the same methods of conjugating the chelants to the dendrimer and metallating the chelants as described above for the diagnostic moieties can be used for the therapeutic moieties.

The diagnostic or therapeutic moieties described above are conjugated to at least one PNA through at least one of the reactive surface groups of the dendrimer or other polymer by conventional chemical coupling techniques, at any location on the PNA oligomer that does not interfere with PNA hybridization to its target nucleic acid sequence. Preferably, the diagnostic or therapeutic moiety is attached to either terminal subunit of the PNA, although conjugation to an internal subunit is not excluded. Techniques for conjugating one or more PNAs to the diagnostic or therapeutic moiety are within the skill in the art. Where more than one PNA is conjugated to the diagnostic or therapeutic moiety, the PNAs can comprise the same or different base sequence. Where the base sequences of the PNAs conjugated to the diagnostic or therapeutic moiety are different, the base sequences can be complementary to target nucleic acid sequences from different RNA transcripts, or can be complementary to multiple target nucleic acid sequences within the same RNA transcript.

The diagnostic or therapeutic moiety can be conjugated directly to a PNA, or can be conjugated to a PNA through one or more linking moieties. Multiple PNAs can be individually conjugated to different reactive groups on a diagnostic or therapeutic moiety. Alternatively, multiple PNAs can be conjugated to each other in series, and then conjugated to a single reactive group on a diagnostic or therapeutic moiety. Multiple PNAs conjugated to each other in series can optionally be separated from each other by one or more linking moieties.

Preferably, the diagnostic or therapeutic moiety is separated from a PNA by a distance of from about 10 to about 30 Å by one or more linking moieties. The linking moiety can comprise any chemical group that is compatible with the diagnostic or therapeutic moiety and PNA, and that does not adversely affect the uptake of the compound or hybridization of the PNA to its target nucleic acid sequence. Suitable linking moieties are discussed above and include —NH(O)C—CH₂CH₂—C(O)O— and —HN—CH₂CH₂—O—CH₂CH₂—O—CH₂C(O)O, or one or more amino acids, such as a stretch of homo-glycine such as (Gly)₄ or 4-amino butyric acid (also known as “Aba”).

A PNA conjugated to the diagnostic or therapeutic moiety comprises a sequence of naturally occurring or non-naturally occurring purine and pyrimidine bases covalently linked by a backbone. The sequence of bases is analogous to the base sequence of a conventional nucleic acid, and is preferably chosen to be complementary to a target nucleic acid sequence within a cell.

The backbone in conventional nucleic acids consists of a series of ribosyl or deoxyribosyl moieties linked by phosphodiester bonds. In PNAs, the sugar backbone is replaced by a backbone substantially comprising a polyamide, polythioamide, polysulfinamide or polysulfonamide. Thus, the PNA can be viewed as a strand of bases covalently bound by linking moieties comprising amide, thioamide, sulfinamide or sulfonamide linkages. Most preferably, the linking moieties in the PNA backbone comprise N-ethylaminoglycine units, and the bases are covalently bound to the PNA backbone by methylene-carbonyl groups. At least some of the purine and pyrimidine bases in a PNA are capable of hydrogen bonding with complementary bases of a target nucleic acid sequence.

Sequences of PNAs are defined by reference to the bases attached to the backbone at a given position. For a given PNA, the nomenclature is modeled after traditional nucleotide nomenclature, identifying each PNA by the identity of its sequence of base such as the heterocycles adenine (A), thymine (T), guanine (G) and cytosine (C). PNAs do not exhibit 5′ to 3′ directionality as do conventional nucleic acids; however, PNA sequences are provided herein in the amino to carboxy orientation. It is understood that a PNA sequence listed in the amino to carboxy orientation is equivalent to a nucleic acid sequence listed in the 5′ to 3′ direction. The nomenclature of conventional nucleic acids that indicates oligomer length is also used herein for the PNAs; thus, a PNA having four bases linked together through a backbone is “four bases” in length.

The PNA portion of the present compounds can be of any length that hybridizes specifically to a target nucleic acid within a cell. For example, the PNA can be from about 8 to about 60 bases in length. Preferably, PNAs can be from about 10 to about 30 bases in length, more preferably from about 12 to about 25 bases in length, particularly preferably from about 12 to about 20 bases in length.

Methods for the preparation and purification of peptide nucleic acids are within the skill in the art, and are described for example in WO 92/20702, WO 92/20703, WO 94/25477, WO 94/28720, WO 95/01370, WO 95/03833, and U.S. Pat. No. 6,180,767, the entire disclosures of which are herein incorporated by reference. Essentially, PNAs are synthesized by adaptation of solution or solid phase peptide synthesis procedures. The synthons are monomer amino acids or their activated derivatives, protected by standard protecting groups.

A PNA oligomer having the preferred backbone; i.e., a backbone formed by N-ethylaminoglycine units, can be formed by linking BOC and Z-protected T, A, C, and G PNA monomers as described in U.S. Pat. No. 6,180,767, supra, which are commercially available from PerSeptive Biosystems (Framingham, Mass.). A suitable solid-phase synthesis of peptide nucleic acids from these BOC and Z-protected monomers is described in Christensen et al., J. Peptide Science 3, 175-183, 1995, the entire disclosure of which is incorporated herein by reference. As an alternative to BOC chemistry, the PNA can be synthesized via FMOC chemistry by linking the FMOC and BHOC-protected T, A, C and G PNA monomers described in U.S. Pat. No. 6,180,767, supra, which are also commercially available from Applied Biosystems (Foster City, Calif.).

The base sequence of the PNA is selected such that the PNA binds to RNA transcripts within a cell. As used herein, an “RNA transcript” is any processed or unprocessed RNA produced from a gene, including heteronuclear RNA (hnRNA) and messenger RNA (mRNA). Production of RNA transcripts from a gene is called “expression.” In preferred embodiments, the PNA binds to mRNA within a cell.

A PNA binds to an RNA transcript within a cell by hybridization to a complementary nucleic acid sequence within the RNA transcript. The complementary nucleic acid sequence within the RNA transcript is called the “target nucleic acid sequence,” and can comprise some or all of a consecutive sequence of bases in the RNA transcript.

Stable duplex formation between a PNA and an RNA transcript depends on the sequence and length of the PNA and the degree of complementarity with the target nucleic acid sequence. Generally, the larger the hybridizing PNA, the more mismatches can be tolerated with the target nucleic acid sequence. One skilled in the art can readily determine the degree of mismatching that can be tolerated between any given PNA and a target nucleic acid sequence based upon the melting temperature (Tₘ) of the resulting duplex, which is taken as the temperature of fifty percent strand dissociation of a PNA/RNA transcript duplex. In general, the PNA used in the present compound will have a base sequence that is completely complementary to a target nucleic acid sequence. However, absolute complementarity is not required, particularly for larger PNAs. Thus, “complementary to” as used herein does not necessarily mean a PNA base sequence has 100% complementarity with the target nucleic acid sequence. Any PNA that can form a stable duplex with the target nucleic acid sequence is considered “complementary to” the target nucleic acid.

The target nucleic acid sequence can be determined by any suitable techniques for obtaining a full or partial sequence of an RNA transcript. Such techniques include production, cloning and sequencing of cDNA or isolation and sequencing of coding regions of a gene. A preferred method of obtaining a target nucleic acid sequence for RNA transcripts is the “polony” PCR colony method, described in Example 1 below and in Butz et al, BMC Biotechnology, 2003, 3:11, the entire disclosure of which is herein incorporated by reference. The polony method can provide sequences of RNA transcripts from multiple mutated genes within a cell, and is particularly suited for obtaining a profile of mutations from an individual patient. K-RAS target nucleic acid sequences obtained from pancreatic cancer cells using the polony method are given in Table 3 below.

In one embodiment, the target nucleic acid sequences comprise sequences of genes implicated in cancer, in particular oncogenes or proto-oncogenes. For example, the target sequence can comprise sequences from RNA transcripts from c-myc (e.g., from hematological, mammary and colorectal malignancies), K-RAS (e.g., from pancreatic, colorectal and pulmonary malignancies), c-myb (e.g., from leukemias, colorectal carcinoma and melanoma), BCR-ABL (e.g., from Philadelphia chromosome-positive leukemias), p53 (e.g., from any tumor type, particularly pancreatic ductal carcinomas), CCND1 (e.g., from pancreatic cancer), and HER2. Other oncogenes and proto-oncogenes such as c-fms, c-kit, c-met, c-trk, c-neu, c-src, c-fes, c-abl, c-fgr, c-yes, c-erbA, c-evi-1, c-gli-1, c-maf, c-lyl-1, c-ets, c-fos, c-jun, c-myb, b-myb, N-myc, L-myc, c-rel, c-vav, c-ski, and c-spi are known to those skilled in the art, and can provide suitable target nucleic acid sequences for purposes of the invention.

The diagnostic or therapeutic moiety of the invention is also conjugated to at least one targeting moiety. In one embodiment, one or more targeting moieties are conjugated, directly to a polymeric diagnostic or therapeutic moiety via one of the dendrimer or other polymer surface reactive groups, or indirectly by conjugation to one or more PNAs that are in turn conjugated to the diagnostic or therapeutic moiety. Multiple targeting moieties, optionally separated by one or more linking moieties, can also be conjugated (directly or indirectly) to reactive group(s) of a diagnostic or therapeutic moiety.

The targeting moiety comprises any chemical substance that is capable of binding to a cell surface molecule or being bound by a cell surface molecule (e.g., a receptor). Binding of the targeting moiety to the cell surface allows the compounds of the invention to be internalized by the cell, for example by receptor-mediated endocytosis, phagocytosis, clathrin-coated pits, or some other internalization mechanism. While the exact mechanism of uptake is not limiting on the scope of the present invention, one preferred mechanism of uptake of the present compounds is receptor-mediated endocytosis. Thus, in one embodiment the targeting moiety is preferably selected such that it is capable of triggering receptor-mediated endocytosis once it is bound to a cell surface. Once internalized by the cell, the compound of the invention is available for binding to target nucleic acid sequences in the cell via the PNA portion of the molecule.

Suitable targeting moieties comprise, for example, a protein, a glycoprotein, a peptide, a steroid, a carbohydrate, a lipid or vitamin capable of binding or being bound by a cell surface molecule and being taken up into the cell. Examples of useful protein-targeting moieties include peptide hormones, antigens, antibodies, growth factors, cytokines, and peptide toxins. The peptide targeting moiety can comprise, for example, 5 to 50 amino acids, more preferably 5 to 30 amino acids, most preferably 5 to 15 amino acids. As used herein, an antibody-targeting moiety includes monoclonal antibodies, chimeric, single chain, and humanized antibodies, as well as Fab fragments retaining substantial antigen-binding ability against a cell surface antigen. Antibody-targeting moieties are particularly useful in the diagnosis and treatment of cancers, which are characterized by the cell surface expression of tumor-specific antigens.

The targeting moiety can also comprise a fragment of a larger peptide that retains the binding properties of the full-length molecule, or a homolog of peptide that binds to or is bound by a cell surface molecule. By “homolog” is meant any peptide that has a sequence identity of at least about 30%, for example about 40%, about 50%, about 60%, about 70%, about 80%, about 90%, about 95%, or about 98%, with respect to a corresponding segment of the reference peptide. Sequence identity can be computed by using the BLASTP and TBLASTN programs that employ the BLAST (basic local alignment search tool) 2.0.14 algorithm with the default settings. See also Altschul et al. (1990), J. Mol. Biol. 215: 403-10 and Altschul et al. (1997), Nucleic Acids Res. 25:3389-3402, the entire disclosures of which are herein incorporated by reference.

Preferred targeting moieties include, for example, the vitamin folate (to take advantage of the natural endocytosis pathway for that molecule; see Leamon and Low, Proc. Natl. Acad. Sci. USA 88, 5572-5576, 1991); the iron-transport protein transferrin (to take advantage of the receptor-mediated uptake of transferrin-iron complexes by actively metabolizing cells; see Wagner et al., Proc. Natl. Acad. Sci. USA 87, 3410-3414, 1990); any of the following substances that facilitate receptor-mediated endocytosis of nucleic acids, such as epidermal growth factor (EGF); platelet-derived growth factors; urogastrone and analogs thereof; thyrotrypsin releasing hormone (TRH); nerve-growth factor (NGF); and any of the various specific viral factors, e.g., a specific viral antigen of the HIV virus specific to the T4-receptor typical of T4 lymphocytes but which can be also be found on other cells (see Maddon et al., Cell 47, 333, 1986); α₂-macroglobulin; thiodothyronine; thrombine; arachidonic acid; transforming growth factor-α (TGF-α); the various heregulins (HRGs); and alpha fetoprotein (AFP), or fragments or homologs of any of the above targeting moieties that are peptides, provided that the fragments or homologs retain the binding properties of the native peptides.

Particularly preferred targeting moieties include IGF1 and Escherichia coli heat-stable enterotoxins (STs); and fragments or homologs thereof that retain the binding properties of the native peptides.

STs are small peptides of 18 or 19 amino acids that bind to specific cell surface receptors located on the intestinal brush border and activate guanylate cyclase, resulting in an increase in the intracellular cyclic guanosine 3′,5′-monophosphate content of the cell. ST receptors are expressed by primary and metastatic human colonic tumors in vivo, with structural and functional characteristics that are similar to those in normal human colon (Carrithers et al., Gastroenterology 107:1653-1661, 1994). Various forms of native ST may be purified from E. coli by methods within the skill in the art (see Dreyfus et al., Infect. Immun. 46:537-543, 1984; Thompson et al., Anal. Biochem. 148:26-36, 1985, the entire disclosure of which is herein incorporated by reference). Fragments and analogs of native ST can be designed and tested for ST-receptor binding activity according to the method of Carrithers et al., supra, and references cited therein (Hugues et al., Biochemistry 30:10738-10745, 1991; Hugues et al., Mol. Pharmacol. 41:1073-1080, 1992; Crane et al., Int. J. Biochem. 25:557-566, 1993; Hakki et al., Biochim. Biophys. Acta 1151:223-230, 1993), the entire disclosures of which are herein incorporated by reference.

IGF1 binds its cognate cell-surface receptor IGFR1. The IGF1/IGFR1 system plays a major role in development and cell cycle progression, and may play a role in the early phase of tumorigenesis. The amino acid sequence of mature IGF1 is given in SEQ ID NO:53, and is described in GenBank record accession no. NM_{—}000618, the entire disclosure of which is herein incorporated by reference. The disulfide-bonded D-peptide of Gly-Cys-Ser-Lys-Ala-Pro-Lys-Leu-Pro-Ala-Ala-Leu-Cys (SEQ ID NO:54) is a homolog of native IGF1 designed by molecular modeling to compete with the native targeting moiety for binding to IGFR1. The disulfide-bonded D-peptide of Cys-Ser-Lys-Ala-Pro-Lys-Leu-Pro-Ala-Ala-Tyr-Cys (SEQ ID NO:55) inhibits the growth of certain cancer cell lines and competes with the natural targeting moiety for binding to IGFR1, and is also an analog of IGFR1. These analogs are described in Pietrzkowski et al., Cancer Res. 52, 6447-6451, 1992, the entire disclosure of which is herein incorporated by reference. Various IGF1 fragments that bind to IGFR1 are disclosed in WO 93/23067 and WO 95/16703, the entire disclosures of which are incorporated herein by reference. These IGF1 fragments, up to 25 amino acids in length, comprise a sequence corresponding to at least a portion of the IGF1 C or D domain.

It is understood that the order in which the PNA and targeting moiety are conjugated to the diagnostic or therapeutic moiety, or their positioning on the diagnostic or therapeutic moiety, is not critical. Therefore, the compound of the invention can comprise a diagnostic or therapeutic moiety that has at least one PNA and at least one targeting moiety conjugated directly to separate surface active groups. The compound of the invention can also comprise a diagnostic or therapeutic moiety conjugated to at least one PNA, which is in turn conjugated to at least one targeting moiety. Alternatively, the diagnostic or therapeutic moiety can be conjugated to at least one targeting moiety, which is in turn conjugated to at least one PNA.

Thus in one embodiment, the compound of the invention comprises formula (I)

X-L₁-Y (I)

wherein:

X is a diagnostic or therapeutic moiety;

L₁ is a chemical bond or at least one linking moiety; and

Y is P-L₂-T or T-L₂-P, in which
P is at least one peptide nucleic acid comprising a base sequence that is complementary to the target nucleic acid sequence;
L₂ is a chemical bond or at least one linking moiety; and
T is at least one targeting moiety.

### Preferably, Y is P-L₂-T.

Where the diagnostic or therapeutic moiety comprises a metal ion or radioactive isotope, the compounds of the invention may be sold labeled with the metal or radioactive isotope or may be sold in an unlabeled form (e.g. a kit) and labeled with the metal or radioactive isotope at the point of use. The phrases “diagnostic moiety” and “therapeutic moiety” are intended to encompass both the labeled and unlabeled forms; thus, “compounds of the invention” are intended to encompass both those compounds in which the diagnostic or therapeutic moiety is complexed with the metal ion or radioactive isotope and those in which it is not.

The targeting moiety can be conjugated to the PNA via a chemical bond or by one or more conventional chemical linking moieties. The selection of the linking moiety will depend primarily on the chemical nature of the targeting moiety. For example, the linking moiety for conjugating the PNA and targeting moiety can comprise an amine or amido group.

The targeting moiety can be conjugated to the PNA at any location on the PNA that does not adversely affect uptake of the compound into the cell or PNA hybridization to the target nucleic acid sequence inside the cell. Suitable conjugation sites on the PNA can be identified by one skilled in the art, and will depend on the mode of interaction of the targeting moiety with its receptor and the chemical nature of the targeting moiety. Preferably, the targeting moiety is conjugated to either terminal subunit of the PNA.

It is preferred that the PNA and targeting moiety are conjugated together by one or more linking moieties. Preferably, the linking moiety separates the targeting moiety from the PNA by a distance of from about 10 to about 30 Å. Suitable linking moieties include those discussed herein and particularly suitable linking moieties include —NH(O)C—CH₂CH₂—C(O)O— and —HN—CH₂CH₂—O—CH₂CH₂—O—CH₂C(O)O, or one or more amino acids, such as a stretch of homo-glycine such as (Gly)₄ or 4-amino butyric acid (also known as “Aba”).

If the targeting moiety is a peptide, the PNA and peptide targeting moiety can be synthesized separately and then conjugated (either with a linking moiety or by a chemical bond) by known reagents suitable for coupling proteinaceous compounds. Preferably, the peptide targeting moiety is synthesized first, followed by synthesis of the PNA as an extension of the peptide targeting moiety. Alternatively, a linking moiety can be included in the chain between the peptide targeting moiety and PNA during synthesis, by incorporating a modified amino acid at the PNA/targeting moiety junction. The modified amino acid can, for example, comprise an appropriate methylene bridge-containing moiety, such as N-∈-FMOC-aminocaproic acid.

Where FMOC chemistry is used to synthesize the PNA, and the targeting moiety is a peptide, the PNA can be readily attached to the amino or carboxy terminus of the peptide targeting moiety. If it is desired to attach the PNA to an internal amino acid residue of the peptide targeting moiety, an ∈-(N-tBOC)-lysine residue could be included in the peptide targeting moiety. After completion of peptide synthesis by FMOC coupling and cleaving of the terminal FMOC group, the ∈-(N-tBOC)-lysine can be deprotected with acid and can serve as the attachment site for BOC coupling of a PNA.

The amino acids used to form a peptide targeting moiety or peptide linking moiety can comprise D- or L-amino acids, or a mixture of both. Preferably, at least one of the amino acids of the peptide is a D-amino acid, which has the effect of enhancing the biological stability of the compound. As used herein, “amino acid” is meant to include both natural and synthetic amino acids. As used herein, “synthetic amino acid” also encompasses chemically modified amino acids, including but not limited to salts, amino acid derivatives (such as amides), and substitutions. Amino acids contained within the compounds of the invention, and particularly at the carboxy- or amino-terminus, can be modified by methylation, amidation, acetylation or substitution with other chemical groups. Additionally, a disulfide linkage may be present or absent in the peptide moieties in the compounds of the invention.

As mentioned above, different synthetic chemistries can be used for the peptide and PNA syntheses. However, where BOC coupling is used for PNA synthesis and FMOC coupling is used for peptide synthesis, the protecting groups for a peptide-targeting moiety (or linking moiety) can be chosen in such a way as to be compatible with BOC coupling and BOC deprotection. Thus, for FMOC peptide synthesis followed by BOC PNA synthesis, FMOC amino-protected amino acids utilized in the peptide synthesis could include appropriate blocking groups on the amino acid side chains. Such fully protected amino acid acids include, for example, FMOC-Cys(MOB)—OH, wherein the native sulfhydryl group is protected by a methoxybenzyl group: FMOC-Lys(Z)—OH, wherein the native ∈-amino group is protected by a phenylmethoxycarbonyl group; and FMOC-Ser(Bzl)-OH, wherein the native hydroxyl group is protected by a benzyl group. Other suitable side chain-protected FMOC amino acids are known to those skilled in the art. Following the completion of the PNA synthesis onto the peptide-targeting moiety and (if desired) linking moiety, the completed PNA-peptide conjugate can be finally deprotected and cleaved from its solid support.

In a preferred embodiment, the PNA, peptide targeting moiety and peptide linking moiety (if any) are synthesized by the same peptide synthesis chemistry; for example, by conventional FMOC chemistry for peptide synthesis. FMOC-PNA subunits are commercially available, for example from Applied Biosystems (Foster City, Calif.).

The invention provides a diagnostic imaging method, in which cells of a subject that contain transcripts comprising a target nucleic acid sequence are contacted with an effective amount of a compound of the invention. In the practice of the diagnostic method, the compound (hereinafter referred to a “diagnostic compound”) preferably comprises a polymeric (e.g., dendrimeric) diagnostic moiety.

As used herein for all methods, a “subject” includes any animal; for example a mammal, bird, reptile or fish. Preferred subjects are mammals; for example primate, rodent, feline, canine, porcine, ovine or bovine mammals. Particularly preferred subjects are primate mammals, such as humans.

Once a cell is contacted with an effective amount of the diagnostic compound, the diagnostic compound binds to cells in the subject via the targeting moiety, and is internalized by the cell. The PNA portion of the diagnostic compound binds to the target nucleic acid sequence inside the cell and retains the diagnostic compound inside the cell. As used herein for all methods, the compound is “retained” inside the cell if the compound remains in the cell longer than a comparable compound that does not have a PNA comprising the complement to the target nucleic acid sequence. One skilled in the art can readily determine the differential retention time between compounds by using a cell culture assay such as is described in Example 4 below. The compound can then be detected within the cell by any suitable imaging technique, wherein the presence of the compound within the cell indicates a pathological state. Preferably, the pathological state is a cancer.

Suitable imaging techniques include magnetic resonance imaging (MRI), scintigriphic imaging (e.g, planar scintigraphy, SPECT or PET), X-ray, gamma camera imaging, ultrasound, or detection of fluorescent or visible light. The choice of an appropriate imaging technique depends on the nature of the diagnostic centers on the diagnostic moiety, and is within the skill in the art. For example, if the diagnostic centers comprise Gd ions, then the appropriate imaging technique is MRI; if the diagnostic centers comprise radionuclides, an appropriate imaging technique is scintigraphy; if the diagnostic centers comprise ultrasound agents, ultrasound is the appropriate imaging technique, etc.

An “effective amount” of a diagnostic compound is an amount sufficient to yield the desired visualization with the particular imaging technique. Generally dosages of from 0.001 to 5.0 mmoles of chelated contrast-producing ion per kilogram of patient bodyweight are effective to achieve adequate contrast enhancement. For most MRI applications, preferred dosages of chelated metal ion will be in the range from 0.02 to 1.2 mmoles/kg bodyweight. For X-ray imaging applications, dosages of from 0.5 to 1.5 mmoles/kg are generally effective to achieve satisfactory X-ray attenuation. Preferred dosages for most X-ray applications are from 0.8 to 1.2 mmoles of the chelated lanthanide or heavy metal/kg bodyweight. For scintigriphic imaging applications, the effective amount is conveniently expressed in terms of radioactivity; e.g., mCi. Generally, an effective amount of a diagnostic compound for scintigriphic imaging is from about 0.01 mCi to about 100 mCi per 70 kg bodyweight, preferably from about 0.1 mCi to about 50 mCi per 70 kg bodyweight.

In the practice of the diagnostic method, the targeting moiety of the diagnostic compound is chosen to bind to a cell of interest, and the PNA portion of the diagnostic compound preferably comprises a predetermined base sequence that binds to a target nucleic acid with in the cell of interest. The ability to choose appropriate targeting moieties and a predetermined PNA base sequence is within the skill in the art, as described in detail above.

Any cell in the subject can be contacted with the diagnostic compound, but in one preferred embodiment the cell is a cancer cell or a cell overexpressing an oncogene or proto-oncogene. For example, the cancer cell contacted with the present diagnostic compound can be primary or metastatic tumor or neoplastic cells in cancers of at least the following histologic subtypes: sarcoma (cancers of the connective and other tissue of mesodermal origin); melanoma (cancers deriving from pigmented melanocytes); carcinoma (cancers of epithelial origin); adenocarcinoma (cancers of glandular epithelial origin); cancers of neural origin (glioma/glioblastoma and astrocytoma); and hematological neoplasias, such as leukemias and lymphomas (e.g., acute lymphoblastic leukemia, chronic lymphocytic leukemia, and chronic myelocytic leukemia).

The cancer cell contacted with the present diagnostic compound can also be primary or metastatic tumor or neoplastic cells from cancers having their origin in at least the following organs or tissues, regardless of histologic subtype: breast; tissues of the male and female urogenital system (e.g. ureter, bladder, prostate, testis, ovary, cervix, uterus, vagina); lung; tissues of the gastrointestinal system (e.g., stomach, large and small intestine, colon, rectum); exocrine glands such as the pancreas and adrenals; tissues of the mouth and esophagus; brain and spinal cord; kidney (renal); pancreas; hepatobiliary system (e.g., liver, gall bladder); lymphatic system; smooth and striated muscle; bone and bone marrow; skin; and tissues of the eye.

The cancer cell contacted with the present diagnostic compound can also be from cancers or tumors in any prognostic stage of development, as measured, for example, by the “Overall Stage Groupings” (also called “Roman Numeral”) or the Tumor, Nodes, and Metastases (TNM) staging systems. Appropriate prognostic staging systems and stage descriptions for a given cancer are known in the art, for example as described in the National Cancer Institute's “CancerNet” Internet website.

In another embodiment, the compounds of the invention are designed to target a cell expressing a nucleic acid of interest which is absent, diminished or not expressed in the presence of a disease or pathological condition, but is present and expressed in normal tissue. In this situation, the compounds of the invention will bind to cells expressing the nucleic acid, but not tissue that does not, allowing identification of abnormal tissue.

As used herein, a cell can be “contacted” with the present compounds by any technique that exposes the cell to the compound. Suitable techniques for contacting a cell in a subject with the present compounds include any enteral or parenteral route of administration. Parenteral administration is preferred. Suitable enteral administration routes include oral and rectal. Suitable parenteral administration routes include intravascular administration (e.g. intravenous bolus injection, intravenous infusion, intra-arterial bolus injection, intra-arterial infusion and catheter instillation into the vasculature); peri- and intra-tissue injection (e.g. peri-tumoral and intra-tumoral injection); subcutaneous injection or deposition including subcutaneous infusion (such as by osmotic pumps); and direct application to a tumor or to tissue surrounding a tumor, for example by a catheter or other placement device (e.g., a suppository or an implant comprising a porous, non-porous, or gelatinous material, a sialastic membrane, or a fiber). It is preferred that subcutaneous injections or infusions be given near a tumor or suspected tumor site, particularly if the tumor or suspected tumor site is on or near the skin.

When injected intravascularly, the present compounds readily extravasate into solid tumors and distribute relatively evenly within the tumor mass, despite the presence of tight junctions between tumor cells, fibrous stroma, and interstitial pressure gradients. Likewise, compounds of the invention administered peri- or intra-tumorally will readily distribute within the tumor mass.

The invention also provides a therapeutic method, in which cells of subject that contain transcripts comprising a target nucleic acid sequence are contacted with an effective amount of a compound of the invention. In the practice of the therapeutic method, the compound (hereinafter referred to as a “therapeutic compound”) preferably comprises a polymeric (e.g., dendrimeric) therapeutic moiety. The transcripts in the cells that comprise the target nucleic acid sequence are characteristic of a pathological state. Preferably, the pathological state is cancer.

As in the diagnostic method above, therapeutic compound binds to the cell via the targeting moiety and is internalized by the cell. The PNA portion of the therapeutic compound binds to the target nucleic acid sequence, and retains the compound inside the cell. However, the presence of the therapeutic compound within the cell inhibits the growth of the cell, or causes death of the cell.

An “effective amount” of a therapeutic compound of the invention is an amount sufficient to inhibit the growth of or kill a cell in the subject. The effective amount of the therapeutic compound administered to a given subject will depend on factors such as the mode of administration, the stage and severity of the tumor being treated, the weight and general state of health of the subject, and the judgment of the prescribing physician.

Generally, an effective amount of therapeutic compound administered to a subject is from about 1 mCi to about 1000 mCi per 70 kg bodyweight, preferably about 10 mCi to about 500 mCi per 70 kg bodyweight, more preferably about 20 mCi to about 100 mCi per 70 kg bodyweight. It is understood that the present therapeutic methods include multiple administrations of the therapeutic compound.

One of ordinary skill in the art can readily determine whether growth of cells targeted by the therapeutic compounds is inhibited, or whether the targeted cells are killed. For example, inhibition of cell growth or induction of cell death can be inferred if the number of cells targeted by the therapeutic compound in the subject remains constant or decreases after administration of the therapeutic compounds. The number of targeted cells in a subject's body can be determined by direct measurement (e.g., calculating the concentration of leukemic or other targeted cells in the blood or bone marrow) or by estimation from the size of a tumor mass. The size of a tumor mass can be ascertained by direct visual observation or by the diagnostic imaging methods discussed above. The size of a tumor mass can also be ascertained by physical means, such as palpation of the tumor mass or measurement of the tumor mass with a measuring instrument such as a caliper.

In the practice of the therapeutic method, the targeting moiety of the therapeutic compound is chosen to bind to a cell of interest, and the PNA portion of the diagnostic compound preferably comprises a predetermined base sequence that binds to a target nucleic acid with in the cell of interest. The ability to choose appropriate targeting moieties and a predetermined PNA base sequence is within the skill in the art, as described in detail above.

In the practice of the therapeutic method, the techniques by which cells in the subject can be contacted with the therapeutic compounds are the same as those for the diagnostic method discussed above. The types of cells in the subject that can be contacted with the therapeutic agent are also the same as those for the diagnostic method discusses above.

The invention also provides a method by which the therapeutic or diagnostic compounds described above can be retained inside a cell. The method comprises contacting the cell with a compound of the invention, such that the targeting moiety binds to the cell surface. The compound is then internalized into the cell, and the PNA binds to its target nucleic acid sequence inside the cell. Binding of the PNA to its target nucleic acid retains the compound within the cell. The cell that is contacted with the present compounds can be in vitro or in vivo. Preferably, the cell that is contacted with the present compounds is a cancer cell, as described above. In the practice of the method for retaining compounds of the invention within a cell, the targeting moiety is chosen to bind to a cell of interest, and the PNA portion of the compound preferably comprises a predetermined base sequence that binds to a target nucleic acid with in the cell of interest. The ability to choose appropriate targeting moieties and a predetermined PNA base sequence is within the skill in the art, as described in detail above.

In the practice of this method, a cell can be “contacted” with the present compounds by any technique that exposes the cell to the compound in vitro or in vivo. Suitable techniques for contacting a cell with the present compounds in vitro include mixing the compounds with the cell culture medium, or placing the compounds directly onto the cells in culture. Suitable methods for contacting a cell in vivo with the present compounds are discussed above for the diagnostic and therapeutic methods.

Preferably, compounds of the invention are retained in cells that overexpress proto-oncogene. As used herein, “overexpression” of a gene means that expression from the gene increased over a basal level of transcription. Overexpression of an oncogene or proto-oncogene can occur through a mutation in a regulatory sequence of the gene, or can occur through amplification of the an oncogene or proto-oncogene (i.e., an increase in oncogene or proto-oncogene copy number). A basal level of transcription of an oncogene or proto-oncogene can readily be determined by one skilled in the art using standard techniques, for example by measuring expression of the an oncogene or proto-oncogene in cells from normal tissue. Oncogene or proto-oncogene expression in target cells can be assayed and compared to the basal level of transcription. Thus, the invention also provides a method of detecting overexpression of an RNA transcript inside a cell. Preferably, the overexpressed RNA transcript that is detected is from an oncogene or proto-oncogene.

In the practice of the method of detecting overexpression of RNA transcripts, the targeting moiety of the diagnostic compound is chosen to bind to a cell of interest, and the PNA portion of the diagnostic compound preferably comprises a predetermined base sequence which binds to a target nucleic acid with in the cell of interest. The ability to choose appropriate targeting moieties and a predetermined PNA base sequence is within the skill in the art, as described in detail above.

Expression of proto-oncogenes in normal and target cells can be determined by conventional molecular biology techniques, such as described in Molecular Cloning: A Laboratory Manual J. Sambrook et al., eds., Cold Spring Harbor Laboratory Press, 2nd ed. 1989. For example, the level of proto-oncogene expression may be determined by probing total cellular RNA isolated from normal and target cells with a complementary probe for the relevant mRNA. The total RNA can be fractionated in a glyoxal/agarose gel, transferred to nylon and hybridized to an appropriately labeled nucleic acid probe for the target mRNA. Relative levels of mRNA expression from the normal and target cells can then be determined, for example by comparing the relative intensity of bands on the gel.

In the methods described above, cells can be contacted with compounds of the invention that have been formulated into pharmaceutical compositions. As used herein, a “pharmaceutical composition” includes compositions for human and veterinary use. Pharmaceutical compositions for parenteral administration are characterized as being sterile and pyrogen-free.

Formulation of the present compounds into pharmaceutical compositions is within the skill in the art; general guidance for preparing such composition can be found, for example, Remington's Pharmaceutical Science, 17th ed., Mack Publishing Company, Easton, Pa. (1985), the entire disclosure of which is herein incorporated by reference.

The present pharmaceutical formulations comprise a compound of the invention and a physiologically acceptable carrier. Preferred physiologically acceptable carriers are water, buffered water, normal saline, 0.4% saline, 0.3% glycine, hyaluronic acid and the like.

Pharmaceutical compositions of the invention can also comprise conventional pharmaceutical excipients and/or additives. Suitable pharmaceutical excipients include stabilizers, antioxidants, osmolality adjusting agents, buffers, and pH adjusting agents. Suitable additives include physiologically biocompatible buffers (e.g., tromethamine hydrochloride), or additions (e.g., 1 to 50 mole percent) of calcium or sodium salts (for example, calcium chloride, calcium ascorbate, calcium gluconate or calcium lactate). The pharmaceutical composition, if desired, can also contain minor amounts of wetting or emulsifying agents, or pH buffering agents. Oral formulations can include standard carriers such as pharmaceutical grades of mannitol, lactose, starch, magnesium stearate, sodium saccharine, cellulose, magnesium carbonate, etc.

The compound of the invention can also be formulated as a neutral or salt form. Pharmaceutically acceptable salts of the present compounds include those formed with free amino groups such as those derived from hydrochloric, phosphoric, acetic, oxalic, and tartaric acids, and those formed with free carboxyl groups such as those derived from sodium, potassium, ammonium, calcium, ferric hydroxides, isopropylamine, triethylamine, 2-ethylamino ethanol, histidine, and procaine.

Particularly for compounds of the invention in which the diagnostic or therapeutic moiety comprises a radionuclide, a single, or multi-vial kit that contains all of the components needed to prepare the compounds (other than the radionuclide), is an integral part of this invention.

A single-vial kit preferably contains a chelating ligand (if a metal radionuclide is used), a source of stannous salt (if reduction is required, e.g., when using technetium), or other pharmaceutically acceptable reducing agent, and is appropriately buffered with pharmaceutically acceptable acid or base to adjust the pH to a value of about 3 to about 9. The quantity and type of reducing agent used would depend highly on the nature of the exchange complex to be formed. The proper conditions are well known to those that are skilled in the art. It is preferred that the kit contents be in lyophilized form. Such a single vial kit may optionally contain labile or exchange ligands such as glucoheptonate, gluconate, mannitol, malate, citric or tartaric acid and can also contain reaction modifiers such as diethylenetriamine-pentaacetic acid (DPTA), ethylenediamine tetraacetic acid (EDTA), or α, β, or γ cyclodextrin that serve to improve the radiochemical purity and stability of the final product. The kit may also contain radiation stabilizers (known to those skilled in the art, and may include, for example, para-aminobenzoic acid, ascorbic acid, gentistic acid and the like), other stabilizers, bulking agents such as mannitol, that are designed to aid in the freeze-drying process, and other additives known to those skilled in the art.

A multi-vial kit preferably contains the same general components but employs more than one vial in reconstituting the radiolabeled compound. For example, one vial may contain all of the ingredients that are required to form a labile Tc(V) complex on addition of pertechnetate (e.g. the stannous source or other reducing agent). Pertechnetate is added to this vial, and after waiting an appropriate period of time, the contents of this vial are added to a second vial that contains the ligand, as well as buffers appropriate to adjust the pH to its optimal value. After a reaction time of about 5 to 60 minutes, the radiolabeled compounds of the present invention are formed. It is advantageous that the contents of both vials of this multi-vial kit be lyophilized. As above, reaction modifiers, exchange ligands, stabilizers, bulking agents, etc. may be present in either or both vials.

The invention will now be illustrated by the following non-limiting examples.

### Example 1

### Characterizing Mutations in Human Pancreatic Cancers

Polymerase colony, or “polony” technology is a form of PCR in which the amplification reaction is immobilized in a thin polyacrylamide gel attached to a microscope slide. As the amplification reaction proceeds, the PCR products diffuse radially within the gel from its immobilized template (e.g., genomic DNA), giving rise to a circular PCR product, also called a “polymerase colony” or “polony”. When the gel is stained with SybrGreen I and scanned with a microarray scanner, the polymerase colony resembles a colony on an agar plate, hence its name. In this experiment, polony technology was used to screen pancreatic cancer cells for somatic mutations in p53 and K-RAS2 genes at mutational hotspots within these two genes.

Polony slide preparation—To preserve the integrity of the polyacrylamide gels used for the polony reactions, Teflon-printed, 24.4×16.7 mm oval slides (Electron Microscope Sciences) were treated with Bind Silane (Amersham) in accordance with the manufacturer's instructions. Initially, the slides were washed for 15 minutes with doubly deionized water containing ammonium formate, pH 3.5. The slides were then removed from the water bath and allowed to dry in a fume hood for 15 to 20 minutes. While the slides were drying, 4 mL of Bind Silane was added to 1 L of doubly deionized water containing ammonium formate, pH 3.5, and allowed to dissolve. Once the Bind Silane/water solution became clear, indicating complete Bind Silane dissolution, the slides were incubated in this solution for about 1.5 hours. The slides were then removed and dried in air prior to storage in a desiccator.

### Preparation of Pancreatic Cell Line Genomic DNA

The human pancreatic cancer cell lines AsPC1, CAPAN-1 and Panc-1 were purchased from the American Type Culture Collection. The cells were grown in Dulbecco's Modified Eagle's Medium (DMEM) with 10% fetal bovine serum at 37° C. media in humidified air containing 5% CO₂. Genomic DNA was harvested from these cells using a Qiagen Blood and Cell Culture DNA Midi Kit.

Casting Polony Gels

Casting polony gels and genotyping mutational hotspots was performed as previously described (Butz et al., 2003, BMC Biotechnol. 3:11). The following master mix recipe was used to cast 12 polony gels. In a microcentrifuge tube, 131.0 μL of filter-sterilized doubly deionized water, 25.5 μL of 10× JumpStart Taq Polymerase Reaction Buffer (Sigma), 2.55 μl, of dNTP (20 mM each), 1.5 μL of 30% BSA (Sigma), 2.55 μL 10% Tween 20, and 56.16 μL of degassed, filter-sterilized 20% acrylamide were combined and vortexed briefly to mix. For each position within a mutational hotspot to be genotyped, 20 μL of master mix was combined with 1 μL of genomic DNA as well as 0.23 μL of each the forward and reverse primers (50 μM; see Table 1) designed to polony amplify the portion of the exon bearing the mutational hotspot(s). Depending on whether the sense or anti-sense strand was to be sequenced, either the forward or reverse primer was modified with a 5′ acrydite, which is necessary to make the polony single stranded (see below).

TABLE 1
Primers used to polony amplify p53 and K-RAS2 exons bearing
mutational hotspots from pancreatic cancer cell line genomic DNA

| Primer Name | Sequence | SEQ ID NO: |
|---|---|---|
| p53 exon5 forward | tgccctgactttcaactctgtctccttcctc | 1 |
| p53 exon5 reverse | ccagacctaagagcaatcagtgaggaatcagaggc | 2 |
| p53 exon7 forward | gttatctcctaggttggctctgactgtacca | 3 |
| p53 exon7 reverse | gtggatgggtagtagtatggaagaaatcggt | 4 |
| p53 exon8 forward | ggtaggacctgatttccttactgcctcttgc | 5 |
| p53 exon8 reverse | gataaaagtgaatctgaggcataactgcacc | 6 |
| kras exon1 forward | tggtggagtatttgatagtgtattaaccttatgtg | 7 |
| kras exon1 reverse | agagaaacctttatctgatatcaaagaatggtcctg | 8 |
| kras exon2 forward | tgaagtaaaaggtgcactgtaataatccagac | 9 |
| kras exon2 reverse | taatgtcagcttattatattcaatttaaacccacc | 10 |

Immediately prior to casting the polony gel, 1.38 μL of JumpStart Taq Polymerase (Sigma), 0.34 μL of 5% APS, and 0.34 μL of TEMED was added to the master mix/primer/DNA solution. The sample was mixed and 20 μL of the mixture pipetted into the void space created by placing a coverslip onto a Teflon-masked, Bind Silane-treated slide. The coverslip was then moved to completely enclose the gel. After letting the polyacrylamide solidify for at least 10 minutes, a hybrid well cover (Grace Bio-labs) was placed on top of the gel and light mineral oil was pipetted into the hybrid well chamber. The slide was placed in a hybridization tower and PCR was performed as follows. Initially the samples are heated to 94° C. for 2 minutes, followed by 39 cycles of 94° C. for 15 seconds, (Tₘ−3)° C. for 30 seconds and 72° C. for 30 seconds, and a final extension step at 72° C. for 2 minutes. The Tₘ is the melting temperature of the PCR primer with the lower melting temperature in a given primer pairing.

Upon completion of the PCR reaction, the hybrid well cover was removed and the slide was placed in hexane for 5 minutes to remove the mineral oil. The coverslip was removed carefully. The slide was then dipped in clean hexane to remove residual oil prior to incubation in a 2×SYBR Green solution (20 μL SYBR Green II (Molecular Probes) in 100 mL 1×TBE) for 15 minutes. Finally, the slide was washed in 1×TBE for 15 minutes to remove non-specific SYBR Green fluorescence prior to scanning the gel using a ScanArray 5000 microarray scanner (Perkin-Elmer) with the FITC laser and filter set.

Denaturation and Electrophoresis of Polony Gels

Prior to genotyping, the double stranded polonies were made single stranded by stripping away the non-acrydited strand in a two-step procedure. First, the polonies were denatured by incubating the gels in 1×SSC, 70% formamide, and 25% doubly deionized-water, at 70° C. for 15 minutes. Immediately following denaturation, the gels were subjected to electrophoresis to remove the non-acrydited strand. To achieve this goal, a standard agarose gel electrophoresis box was used as follows. Both the negative and positive electrode reservoirs were half filled with electrophoresis buffer (42% urea in 0.5×TBE) and the polony slides were placed on the gel platform. For each gel, Whatman filter paper was cut into two 0.75 inch strips, wetted with electrophoresis buffer, and laid down to connect each reservoir with the end of the gel closest to that reservoir. The gel surface was wetted with buffer and then covered with a standard glass slide to prevent the sample from drying. With the bridge complete, the gel was subjected to 140 V for 2.5 hours.

Hybridization and Single Base Extension

After electrophoresis, the polony slides were washed 4× in Wash1E (0.1 M Tris-HCl, pH 7.5, 20 mM EDTA, 0.5 M KCl) to prepare for hybridization of the sequencing primer. Two hundred microliters of annealing buffer (6×SSPE, 0.01% Triton X-100) containing 0.5 primer (Table 2) was then pipetted onto the gel and covered with a hybrid well chamber. The sample was then placed in a hybridization tower and heated for 2 minutes at 94° C. followed by 20 minutes at (Tm −3)° C. to facilitate hybridization.

Genotyping of mutational hotspots was accomplished by performing single base extensions of the hybridized sequencing primer with fluorescently labeled deoxynucleotides. Following hybridization, the gels were washed 2× in Wash1E and then equilibrated in Klenow extension buffer (50 mM Tris-HCl, pH 7.5, 5 mM MgCl₂, 0.01% Triton X-100) for 1 minute. For each sample, 50 μL solution containing approximately 1 unit of Klenow large fragment (New England Biolabs), 3 μg of single stranded binding protein (US Biochemicals), and 0.5 μM Cy3- or Cy5-labeled dATP, dCTP, dGTP, or dUTP (Perkin-Elmer) was pipetted onto the gel. The single base extension was allowed to proceed for 2 minutes. The gels were then washed in Wash1E to reduce background fluorescence and scanned on the ScanArray5000 with the appropriate lasers and filters. The process of formamide denaturation, hybridization, extension, and scanning was repeated 3 additional times for each primer in order to do a single base extension with each of the four labeled nucleotides. This was necessary to completely genotype each nucleotide position within a mutational hotspot.

TABLE 2
Primers used to sequence codons in p53 and
K-RAS2 that experience a high incidence of
mutation during carcinogenesis

| | | SEQ |
|---|---|---|
| | | ID |
| Primer Name | Sequence | NO: |
| p53 c175 pos1 for | gcacatgacggaggttgtgagg | 11 |
| p53 c175 pos2 for | gcacatgacggaggttgtgaggc | 12 |
| p53 c175 pos3 for | gcacatgacggaggttgtgaggcg | 13 |
| p53 c175 pos3 rev | cagcgctcatggtggggggca | 14 |
| p53 c175 pos2 rev | cagcgctcatggtggggggcag | 15 |
| p53 c245 pos1 for | gtaacagttcctgcatgggc | 16 |
| p53 c245 pos2 for | gtaacagttcctgcatgggcg | 17 |
| p53 c245 pos3 for | gtaacagttcctgcatgggcgg | 18 |
| p53 c248 pos1 for | cctgcatgggcggcatgaac | 19 |
| p53 c248 pos2 for | cctgcatgggcggcatgaacc | 20 |
| p53 c248 pos3 for | cctgcatgggcggcatgaaccg | 21 |
| p53 c249 pos3 rev | gtgatgatggtgaggatggg | 22 |
| p53 c249 pos2 rev | gtgatgatggtgaggatgggc | 23 |
| p53 c249 pos1 rev | gtgatgatggtgaggatgggcc | 24 |
| p53 c273 pos1 for | gacggaacagctttgaggtg | 25 |
| p53 c273 pos2 for | gacggaacagctttgaggtgc | 26 |
| p53 c273 pos3 for | gacggaacagctttgaggtgcg | 27 |
| p53 c282 pos1 for | gtgcctgtcctgggagagac | 28 |
| p53 c282 pos2 for | gtgcctgtcctgggagagacc | 29 |
| p53 c282 pos3 for | gtgcctgtcctgggagagaccg | 30 |
| kras c12 pos1 for | aacttgtggtagttggagct | 31 |
| kras c12 pos2 for | aacttgtggtagttggagctg | 32 |
| kras c12 pos3 for | aacttgtggtagttggagctgg | 34 |
| kras c13 pos3 rev | gtcaaggcactcttgcctac | 35 |
| kras c13 pos2 rev | gtcaaggcactcttgcctacg | 36 |
| kras c13 pos1 rev | gtcaaggcactcttgcctacgc | 37 |
| kras c61 pos1 for | atattctcgacacagcaggt | 38 |
| kras c61 pos2 for | atattctcgacacagcaggtc | 39 |
| kras c61 pos3 for | atattctcgacacagcaggtca | 40 |

The designations “for” and “rev” indicate whether the anti-sense or sense strand was sequenced, respectively.

Codons 175, 245, 248, 249, 273, and 282 in p53, and codons 12, 13, and 61 in K-RAS2 were sequenced in the genomic DNA of various pancreatic cell lines as follows. Initially, each exon bearing a mutational hotspot was individually PCR amplified in a polyacrylamide gel giving rise to one polony per copy of genomic p53 or K-RAS DNA. The non-acrydited strand of the polony was then stripped away after formamide treatment and electrophoresis. A sequencing primer was hybridized to the single-stranded copy of the PCR-amplified p53/K-RAS2 fragment and a single base extension with either a Cy-3 or Cy-5 labeled dNTP was performed prior to scanning on a microarray scanner. The process of formamide denaturation, hybridization, and extension was repeated three additional times in order to perform an extension with each of the four dNTPs and completely sequence each position.

When all the mutational hotspots were sequenced in cell line Panc-1 (results of sequencing in Table 3), it was determined that K-RAS2 was heterozygous (i.e., one mutant and one wild type allele) at the second position of codon 12, and p53 harbored a mutation at the second position of codon 273 (see also Butz et al., 2003, BMC Biotechnol. 3:11, the entire disclosure of which is herein incorporated by reference). In addition to the cell line Panc-1, K-RAS2 mutations in the second position of codon 12 were also shown to be present in the cell lines AsPC1 (G→A) and CAPAN-1 (G→T). These results are in agreement with previously published data concerning the genotype of these cell lines (ATCC).

TABLE 3
Results from sequencing p53 and K-RAS2
mutational hotspots in Panc-1 genomic
DNA.

| codon | strand sequenced | wt | Panc-1 |
|---|---|---|---|
| K-ras | | | |
| 12 | anti-sense | GGT | G G/A T |
| 13 | sense | CCG | CCG |
| 61 | anti-sense | CAA | CAA |
| p53 | | | |
| 175 | anti-sense | CGC | CGC |
| 245 | anti-sense | GGC | GGC |
| 248 | anti-sense | CGG | CGG |
| 249 | sense | TCC | TCC |
| 273 | anti-sense | CGT | CAT |
| 282 | anti-sense | CGG | CGG |

Polony amplification of genomic DNA from strains with equal p53 and K-RAS2 copy numbers yielded equivalent numbers of p53 and K-RAS polonies. This eliminates the role of primer bias contributing to the distinct number of p53 and K-RAS polonies amplified in Panc-1 genomic DNA. For example, Panc-1 was determined to possess only one copy of p53 that possessed an intragenic mutation in codon 273, and two copies of K-RAS (one wildtype and one with an intragenic mutation in codon 12). These results are consistent with findings from previous work.

### Example 2

### Preparation of Dendrimer-Pna-Peptide Diagnostic or Therapeutic Compounds

### Solid Phase Synthesis of the Protected H₂N-Spacer₂-PNA-Spacer₂-Peptide on Polystyrene Resin.

Spacer₂ is —HN—CH2CH2-O—CH2CH2-O—CH2C(O)O—, PNA is —HN-GCCAACAGCTCC—C(O)O— (where GCCAACAGCTCC is the nucleic acid sequence SEQ ID NO:43), and the peptide targeting moiety (“Peptide”) is —HN-Cys-Ser-Lys-Cys-(SEQ ID NO:41).

The peptide-targeting moiety was assembled by Fmoc-protected monomer coupling on a NovaSyn TGR resin (loading, 0.2-0.3 mmol/g) (Novabiochem) on an Applied Biosystems 430A peptide synthesizer. Then, PNA monomers were sequentially coupled to the resin on the 8909 DNA synthesizer, using the Fmoc-chemistry protocol for the peptide amino acids. After each coupling of a peptide nucleic acid monomer, the quantity of Fmoc groups released was measured to determine the yield of coupling. According to Fmoc quantitation at 301 nm, the average yield of coupling reactions was 85-92%. Typical UV absorption spectra for detection of Fmoc groups were obtained for each step of coupling. The specific 9-piperidino-dibenzofulvene breakdown product of Fmoc absorbs at 301 nm with ∈=7780/M·cm. A Spacer₂ was added to the chain just before the first PNA monomer, and again after the last PNA monomer. After assembly of the Spacer₂-PNA-Spacer₂-peptide on the polymer support, cyclization on a resin, cleavage, and deprotection of spacer-PNA-peptide was performed.

### Synthesis of the HOOC-Spacer₁-Spacer₂-PNA-Spacer₂-Peptide

Spacer₁ is —NH(O)C—CH2CH2-C(O)O—. Solid phase conjugation of Spacer₁ to Spacer₂-PNA-Spacer₂-Peptide was performed by conjugation H₂N-Spacer₂-PNA-Spacer₂-Peptide with succinic anhydride on polystyrene resin. After conjugation of Spacer₁ to Spacer₂-PNA-Spacer₂-Peptide on polymer support, the cyclization on a resin and deprotection of HOOC-Spacer₁-Spacer₂-PNA-Spacer₂-peptide was performed.

Oxidation and cyclization of S-groups on a resin. Cleavage and Deprotection of HOOC-Spacer₁-Spacer₂-PNA-Spacer₂-Peptide.

HOOC-Spacer₁-Spacer₂-PNA-Spacer₂-peptide-resin was suspended in (Me)₂NCHO. Oxidation was carried out with I₂ (0.1 M) for 4 hours at room temperature. The resin was washed with (Me)₂NCHO to remove excess iodine and dried in a vacuum. Cleaved and deprotected PNA-Peptides were purified by preparative RP-HPLC at 50° C. and gave an overall final yield of 17%. Preparative C₁₈ HPLC of a crude mixture of HCOO-Spacer₁-Spacer₂-PNA-Spacer₂-Peptide was performed on a 10×250 mm Alltima C₁₈ column by eluting with a 5% to 70% CH₃CN gradient over 25 minutes in aqueous 0.1% CF₃CO₂H, at 1 mL/min. at 50° C., and monitored at 260 nm.

### Synthesis of the Diagnostic and Therapeutic Moieties

Fluorescent Dendrimers—Free PAMAM dendrimers typically do not have any UV absorbance, and it is not possible to detect PAMAM dendrimers and their derivatives during purification by HPLC. A PAMAM generation 3 dendrimer was therefore synthesized and labeled with Alexa Fluor 555 dye succinimidyl ester (Molecular Probes, Eugene Oreg.). The final Alexa Fluor 555 PAMAM conjugate was separated from the free Alexa Fluor 555 dye by filtration on Centricon YM-3. The upper fraction after Centricon YM-3 filtration displayed a typical Alexa Fluor 555 dye spectrum with lambda-max at 555 nm, and the lower fraction consisted of free Alexa Fluor 555 dye and gave a spectrum with lambda-max at 552 nm. The molar ratio of Alexa Fluor 555 in upper vs. lower fraction was 96:4, which implies 96% yield of labeling of PAMAM. The purified Alexa Fluor 555-PAMAM(3G) conjugate, 1.1 A555 unit in 200 mL 0.1% CF₃CO₂H, was analyzed by reverse phase HPLC and eluted at 13 minutes.

MR Active or Radioactive Dendrimers—Polyamidoamine (PAMAM) generation 3 (32 amino groups) or generation 6 (256 amino groups) are synthesized by standard techniques. After conjugation of the dendrimers to the HOOC-Spacer₁-Spacer₂-PNA-Spacer₂-peptide (see below), the chelant DTPA is conjugated to the remaining 31 or 255 free surface groups of the dendrimers, and the DTPA is metallated with either Gd or ¹⁸⁸Re.

### Assembly of the Diagnostic or Therapeutic Compound

HOOC-Spacer₁-Spacer₂-PNA-Spacer₂-peptide is conjugated to the fluorescent, MR active or radioactive PAMAM dendrimers by the free carboxyl group on Spacer₁, to form diagnostic or therapeutic compounds of the formula:

PAMAM-Spacer₁-Spacer₂-PNA-Spacer₂-Peptide-C(O)—NH₂

### Example 3

### Small Angle X-Ray Scattering Modeling of Gd₃₁-Dendrimer-PNA-Peptide Conjugates

Small angle x-ray scattering modeling calculations of the motions of the Gd₃₁-dendrimer-PNA-peptides in water and dimethylformamide have been performed as described in Prosa et al., J. Polymer Sci. Part B: Polymer Physics, 1998, 17:2913-2924, and predict good accessibility of the PNA probe to solvent.

The kinetic and potential energy of PAMAM generation 3 with 32 amines was calculated in dimethylformamide (DMF.) at 300° K for 5×10⁵ steps of 1 fsec, for a total of 500 psec, to determine the minimum energy configuration at thermal equilibrium. The DMF medium was simulated by applying the dielectric constant of DMF (∈=36.647). The pair correlation function showed that the modeled amine endgroups were folded into the PAMAM(3G) dendrimer, with a high likelihood of finding amino endgroups close to the center carbons. Yet, there was a high probability of finding endgroups in the range of 15 Å to 20 Å. Overall, the model indicates that the dendrimer will have a globular, spherical structure in DMF.

A run of 3×10⁵ steps for PAMAM-3 in water (∈=80) at 300° K was also performed. The pair correlation function indicates that most of the endgroups seem to be folded back into the dendrimer. Therefore, the molecule is likely even more of a spherical globule in water than in DMF.

A 5×10⁵ step run of PAMAM-3 with Spacer₁ attached to one of the amine endgroups in DMF was performed. The pair correlation function showed a shift of amine endgroups away from the center, with the probability of finding an endgroup shifting out to approximately 13 Å. The attachment of the spacer indicated that it stretched out the arm of the dendrimer to which it was attached, and furthermore allowed the dendrimer more degrees of freedom. The overall shape of the dendrimer was changed away from a spherical object.

Comparing all the pair correlation functions together clearly showed that the lower dielectric constant allowed the endgroups more freedom to move away from the dendrimer center. Furthermore, the comparison showed that the attachment of a molecule to a dendrimer endgroup allowed that endgroup to move away from the dendrimer center.

The kinetic and potential energy of the K-RAS PNA antisense 12-mer in a run of 1×10⁶ steps in water at 300° K was also calculated as above for the dendrimer compounds. This run was performed to relax the initial molecule, yielding a prediction of an extended PNA structure.

The simulations discussed above predict no barriers to dendrimer-PNA-targeting moiety synthesis in organic solvents comparable to dimethylformamide, or to utilization of such compounds in aqueous environments such as a cell.

### Example 4

### Uptake of PNA-Peptide Conjugates by Tumor Cells In Vitro

To improve cellular uptake of an IGFR1 antisense sequence targeted against IGFR1 mRNA codons 706-709 (CCGCTTCCTTTC, SEQ ID NO:42; Ullrich et al., 1986, EMBO J. 5:10:2503-2512), a PNA with this base sequence was conjugated to a D-amino acid IGF1 peptide having the sequence (Gly)₄D(Cys-Ser-Lys-Cys). This peptide binds selectively to the cell surface receptor for insulin-like growth factor 1 (IGFR1), which is overexpressed on malignant cells (Pietrzkowski et al., 1992, Mol. Cell. Biol. 12:9:3883-3889). The same PNA was also conjugated to a control peptide of the sequence (Gly)4D(Cys-Ala-Ala-Cys), which is not expected to bind to cells expressing IGFR1. The IGF1 D-peptide and control peptide was assembled on (4-methyl benzhydrypamine (MBHA) resin, and then the PNA was extended as a continuation of the peptide. The IGF1 peptide and control peptide sequences were radiolabeled with ¹⁴C or fluorescently labeled with fluorescein isothiocyanate (Basu & Wickstrom, 1997, Bioconj. Chem. 8:4:481-488).

Cellular uptake of the PNA-peptide conjugate Gly-CCGCTTCCTTTC-(Gly)₄D(CysSerLysCys), the control Gly-CCGCTTCCTTTC-(Gly)₄D(CysAlaAlaCys), and a control Gly-CCGCTTCCTTTC PNA without the peptide segment, were studied in three cell lines: murine BALB/c 3T3 cells, which express low levels of murine IGFR1; p6 cells, which are BALB/c 3T3 cells overexpressing a transfected human IGFR1 gene; human Jurkat cells, which do not express IGFR1, as a negative control.

Results

Denaturing SDS gel electrophoresis and MALDI-TOF mass spectroscopy results were consistent with the chimeric sequence. The IGFR1-specific Gly-CCGCTTCCTTTC-(Gly)₄D(CysSerLysCys) conjugate displayed much higher uptake than the control Gly-CCGCTTCCTTTC, but only in cells expressing IGFR1, measured with both the ¹⁴C-conjugate and the fluoresceinyl-conjugate (Basu & Wickstrom, 1997, Bioconj. Chem. 8:481-488) (GlyGlyGlyGly, i.e., Gly₄, is SEQ ID NO:33). This indicates that antisense PNAs conjugated to a targeting moiety can be delivered to and internalized by specific cells in vitro.

### Example 5

### Targeting Cell Surface Receptors in Tumor Cells with Dendrimer-PNA-Peptide Conjugates In Vivo and In Vitro

In this prophetic example, the ability of Gd₂₅₆-PNA-peptide compounds to target IGFR1 receptors can be evaluated for cultured Panc1 or AsPC1 human pancreatic cancer cells, and for cultured MCF7M and BT474 human breast cancer cells, as follows.

Construction of Gd₂₅₆-Dendrimer-PNA-Peptides

Gd₂₅₆-dendrimer-PNA-peptides capable of binding to the cell surface receptor for IGF1 are prepared as described above, with the PNA portions comprising base sequences that hybridize specifically to mRNAs for the following oncogenes: activated K-RAS mutated in the 12th codon, CCND1, HER2, MYC, and mutant tumor suppressor p53 (see Table 4). The Gd₂₅₆-dendrimer PNA-peptide compounds have the formula:

Gd₂₅₆-dendrimer-Spacer₁-Spacer₂-PNA-(Gly)₄D(Cys-Ser-Lys-Cys)

The structures of Spacer₁ and Spacer₂ are presented in Example 2. The PNA antisense and mismatch (control) sequences are given Table 4.

The PNA-peptide portions of the compounds are assembled by solid phase synthesis (Tian & Wickstrom, Organic Letters 4, 4013-6, 2002), beginning from the C-terminus. First the IGF1 D-peptide analog D-CSKC is extended from a NovaSyn TGR resin, followed by a Gly₄ spacer, using Fmoc coupling, followed by the PNA sequences, and cyclized on column before cleavage, as described above (and see Basu & Wickstrom, 1997, Bioconj. Chem. 8:481-488 and Good & Nielsen, 1997, Antisense & Nucleic Acid Drug Dev. 7:4:431-437, the entire disclosures of which are herein incorporated by reference).

For critical analysis of sequence dependence, control PNA sequences include 4 central mismatches to preclude antisense hybridization. Homogeneity is analyzed by electrophoresis on SDS-PAGE gels (Basu & Wickstrom, 1997, Bioconj. Chem. 8:481-488) and by capillary electrophoresis on open capillaries under peptide conditions. Molecular masses are determined by electrospray or MALDI-TOF mass spectroscopy (Basu & Wickstrom, 1997, Bioconj. Chem. 8:481-488). The criterion for adequate purity is 95%.

TABLE 4
K-RAS, CCND1, HER2, MYC, and p53 antisense and
mismatch PNA sequences

| | | |
|---|---|---|
| K-RAS antisense | 5′-GCCAACAGCTCC (43) | codons 10 to 13 |
| K-RAS mismatch | 5′-GCCTTGTGCTCC (44) | 4 central mismatches |
| CCND1 antisense | 5′-CTGGTGTTCCAT (45) | codons 1 to 4 |
| CCND1 mismatch | 5′-CTGGACAACCAT (46) | 4 central mismatches |
| ERBB2 antisense | 5′-CATGGTGCTCAC (47) | codons −3 to 1 |
| ERBB2 mismatch | 5′-CATGCACTTCAC (48) | 4 central mismatches |
| MYC antisense | 5′-GCATCGTCGCGG (49) | codons −3 to 1 |
| MYC mismatch | 5′-GCATGTCTGCGG (50) | 4 central mismatches |
| p53 antisense | 5′-CCCCCTGGCTCC (51) | exon 10 |
| p53 mismatch | 5′-CCCCTACCCTCC (52) | 4 central mismatches |

The numbers in parentheses represent SEQ ID NOS:

The T1 value of water of the cultured pancreatic and breast tumor cells treated with the Gd₂₅₆-dendrimer-PNA-peptides is measured to determine if T1 increases in the case of cell-specific peptides and oncogene-specific PNAs. If at least 90% pure probes are not obtained after single chromatographic purification, variations are reiterated in coupling protocols for the PNA-peptide with the dendrimer to increase coupling yields. It is expected that the probes will consistently display at least a 3-fold excess of gene-specific probes and a 3-fold increase in T1 in cultured cells compared with control sequences.

Cell Targeting Experiments

Pancreatic or breast cancer cells are grown in DMEM with 10% fetal bovine serum, 50 U/mL penicillin, and 50 μg/mL streptomycin in a humidified incubator at 37° C. in 5% CO₂ and 95% air. When the cells reach confluency, they are detached with trypsin/EDTA under standard trypsinization conditions. Cells are sedimented at 450×g for 5 minutes, washed with HBSS and then resuspended in DMEM. Cell titer is determined using a hemacytometer, and cell viability is determined using trypan blue exclusion. Cell titer is then adjusted to 2×10⁷ cells/mL. In each of 6 siliconized 0.5 mL glass test tubes, 10⁷ cells are dispensed in 0.5 mL.

¹⁴⁷Gd₂₅₆-dendrimer-PNA-peptide preparations (specific activity 1.8-5.4 Ci/mmol, with unbound ¹⁴⁷Gd₂₅₆-dendrimer and PNA-peptide each <2%) are diluted and added to each test tube in such a way that the final concentration of PNA-peptide is 10⁻⁷ M to 10⁻¹² M with 10-fold decrements in each subsequent test tube. The final volume in each test tube is rendered constant. Test tubes are then stoppered and placed in a water bath at 37° C. for 2 hours, with gentle mixing every few minutes. The cells are sedimented at 450×g for 5 minutes, and the supernatant is separated and saved. The cells are washed once with 1 mL DMEM, sedimented again, and the supernatants are combined. Radioactivity bound to the cells or remaining in the supernatant is then counted in the scintillation counter. Assays are performed in triplicate. Bound to free ratios (B/F) are determined, then Munsen's SCAM′ ligand binding curves is plotted using the average of the B/F ratios versus log (total ligand concentration added). Kd (or IC₅₀) is the molar concentration at which 50% of the maximum binding occurs.

### Example 6

### Inhibition of Proliferation of Pancreatic and Breast Tumor Cell Lines by Dendrimer-PNA-Peptide Conjugates

In this prophetic example, the ability of Re₂₅₆-PNA-peptide compounds to inhibit proliferation of pancreatic and breast cancer cell lines can be evaluated for cultured Panc1 or AsPC1 human pancreatic cancer cells, and for cultured MCF7M and BT474 human breast cancer cells, as follows.

Panc 1 and AsPC1 human pancreatic cancer cells containing an activating mutation in K-RAS are grown as described above. MCF7M or BT474 human breast cancer cells are also grown as described above. Aliquots of 1×10⁵ cells are plated in 6-well plates, and are allowed to adhere to plates for 24 hours prior to oligonucleotide lipofection. Generation 6 dendrimer-PNA-conjugates targeted to IGF1 are prepared as described above, except that the dendrimer carries ¹⁸⁸Re instead of Gd. The PNA portion of the ¹⁸⁸Re-dendrimer-PNA-peptide conjugates has either the K-RAS antisense PNA base sequence or the mismatch (control) K-RAS sequence from Table 4 above.

¹⁸⁸Re-dendrimer-PNA-peptide conjugates are administered to cells to a final concentration of 0.1, 1.5, or 10.0 μM for 16 hours, after which the medium is removed and replaced with fresh medium, as previously described (Vaughn et al., 1995, Proc. Natl. Acad. Sci. USA 92:8338-8342). The cells are then allowed to grow for about 6 days, because Ras proteins exhibit a half-life of 20 hours (Ulsh and Shih, 1984, Mol. Cell. Biol. 4:1647-1652). Then, cells are washed twice with phosphate-buffered saline, trypsinized, and counted. Viability is determined by the trypan blue dye exclusion assay. Statistical analysis is carried out by applying the Kruskal-Wallis test in InStat 2.01 for Macintosh.

In other experiments, ¹⁸⁸Re-dendrimer-PNA-peptide conjugates comprising antisense or mismatch (control) sequences to CCND1, HER2, MYC, or p53 are tested for their effect on pancreatic and breast tumor cell proliferation.

Alexa Fluor 555 labeled dendrimer-PNA-peptide conjugates analogous to the ¹⁸⁸Re-dendrimer-PNA-peptide conjugates are also prepared and administered to the cells as described above, for visualization purposes to determine whether the conjugates are internalized by cells in vitro.

### Example 7

### Dendrimer-PNA-Peptide In Vivo Imaging and Tissue Distribution Studies

In this prophetic example, the ability of Gd₂₅₆-PNA-peptide compounds to image pancreatic and breast cancer xenografts can be evaluated for cultured Panc1 or AsPC1 human pancreatic cancer cells, and for cultured MCF7M and BT474 human breast cancer cells, as follows.

Oncogene-specific and control Gd256-dendrimer-PNA-peptide compounds are prepared as in Example 5 above, and are administered intravenously to cohorts of nude mice bearing human pancreatic or breast cancer xenografts. BALB/c/nu/nu (nude) mice bearing tumor xenografts are prepared as described in Wickstrom, E., and Tyson, F. L. Differential Oligonucleotide Activity in Cell Culture Versus Mouse Models. Oligonucleotides As Therapeutic Agents, 124-37. Ciba Foundation Symposia, 1997, the entire disclosure of which is herein incorporated by reference. The sensitivity and specificity of magnetic resonance imaging of the targeted oncogene mRNAs in the tumors is determined, relative to the nonspecific signals expected in the liver, gall bladder, and kidneys. The imaging results are compared with radioactive [¹⁴⁷Gd]Gd256-dendrimer-PNA-peptide tissue distribution measurements, and with real time QRT-PCR measurements of the oncogene mRNAs in tumor cells removed from the animals.

Imaging Studies

A pre-determined quantity of ¹⁴⁷Gd bound to dendrimer-PNA-peptide is administered to groups of five mice bearing each through a lateral tail vein. At 15 min, 30 min, and 1, 2, 4, 8, 16, and 24 hours post-injection, mice are lightly anesthetized and imaged using a Starcam (GE, Milwaukee, Wis.) gamma camera equipped with a parallel hole collimator. For images, 300,000 counts are recorded on a paper plate. Mice are then killed in a halothane gas chamber and tissues are dissected. Dissected tissues are washed free of any blood, blotted free of liquid, weighed, and radioactivity associated with each tissue is counted in an automatic gamma counter (Packard Series 5000, Meridien, Conn.), together with a standard radioactive solution of a known quantity of radioactivity prepared at the time of injection. Results are expressed as percent of injected dose per gram of tissue (% I.D./g). Data are evaluated statistically using Student's t test.

¹⁴⁷Gd-dendrimer-PNA-peptide internalization by tumor cells Tumor xenograft samples are disrupted to single cell suspensions, washed, then lysed with a biomaterial fluor cocktail and counted in a liquid scintillation spectrometer. This determines whether tumor cells in a tumor behave similarly to tumor cells in cell culture.

Correlative measurements of oncogene mRNA expression Tumor xenografts are implanted in animals not receiving radioactive ¹⁴⁷Gd-labeled probes. Parallel samples of tumors, livers, gallbladders and kidneys removed at the same time that gamma imaging is performed are correlated with QRT-PCR analysis. This determines mRNA levels in tumors and normal tissues and allows direct comparison of tumor imaging results with tumor mRNA levels.

### Example 8

### Inhibition of Tumor Xenograft Growth in Nude Mice with Dendrimer-PNA-Peptide Conjugates

In this prophetic example, the ability of Re₂₅₆-PNA-peptide compounds to inhibit tumor growth of pancreatic and breast cancer xenografts can be evaluated for cultured Panc1 or AsPC1 human pancreatic cancer cells, and for cultured MCF7M and BT474 human breast cancer cells, as follows.

¹⁸⁸Re-dendrimer-PNA-peptide conjugates with the K-RAS antisense and mismatch (control) PNA base sequences, prepared as in Example 6 above, are utilized to inhibit the growth of pancreatic and breast xenograft tumors in 6-8 week old female athymic nude mice. ¹⁸⁸Re-dendrimer-PNA-peptide conjugates comprising antisense or mismatch (control) PNA base sequences to CCND1, HER2, MYC, or p53 (see Table 4) are also tested for their ability to inhibit tumor growth.

Nude mice bearing pancreatic or breast tumor xenografts on one flank are prepared as described above. ¹⁸⁸Re-dendrimer-PNA-peptide conjugates (or with 5-fluorouracil as a positive control) are injected subcutaneously into the contralateral flank or intraperitoneally on day zero. Six doses of ¹⁸⁸Re-dendrimer-PNA-peptide conjugates or 5-fluorouracil are then administered over a two-week period. Tumor volumes are measured with Vernier calipers in two orthogonal directions three times weekly. Experiments are terminated after about 22 days. Tumor volumes are calculated with the formula: V=1×w²/2.

Correlative measurements of oncogene mRNA expression Tumor xenografts are implanted in animals not receiving radioactive Re¹⁸⁸-labeled probes. Parallel samples of tumors, livers, gallbladders and kidneys removed at the same time that gamma imaging is performed, and oncogene expression levels in the tissues are determined by QRT-PCR analysis. This allows direct comparison mRNA levels in Panc 1 tumors and AsPC1 tumors and normal tissues with tumor imaging results.

All documents referred to herein are incorporated by reference. While the present invention has been described in connection with the preferred embodiments and the various figures, it is to be understood that other similar embodiments may be used or modifications and additions made to the described embodiments for performing the same function of the present invention without deviating therefrom. Therefore, the present invention should not be limited to any single embodiment, but rather should be construed in breadth and scope in accordance with the recitation of the appended claims.

We claim:

1. A compound comprising a polymeric diagnostic moiety (X) covalently conjugated to an antisense peptide nucleic acid (PNA) (P), covalently conjugated to a targeting moiety (T) that selectively binds to a cell surface receptor, wherein the PNA comprises a base sequence that is complementary to a target nucleic acid sequence, or pharmaceutically acceptable salts thereof, provided that the compound is represented by a formula
X—S1-P—S2-T
or pharmaceutically acceptable salts thereof, wherein S1 and S2 represent flexible, hydrophilic spacer moieties from about 10 Å to about 30 Å, provided that S1 is covalently bound to X and P, and S2 is covalently bound to P and T.

2. The compound of claim 1, wherein the diagnostic moiety (X) comprises a branched oligomeric polychelant complexed to one or more diagnostic metal ions such as a paramagnetic metal ion, a heavy metal ion, or an ion of a radioactive metal isotope.

3. An antisense diagnostic imaging method, comprising:
(a) contacting cells of a subject that contain transcripts comprising a target nucleic acid sequence with a compound of claim 1, such that the compound binds to the cells via the targeting moiety (T) and is internalized by the cell;
(b) allowing the antisense PNA (P) to bind to the target nucleic acid sequence and retain the compound inside the cell; and
(c) detecting the compound within the cells by means of the diagnostic moiety (X).

4. The method of claim 3, wherein the compound is detected within the cells by magnetic resonance imaging, scintigraphic imaging, X-ray, gamma camera imaging, positron emission tomography, ultrasound, or detection of fluorescent or visible light.


SEQUENCE LISTING (55 sequences)

SEQ ID NO: 1 - DNA, 31 bp, Artificial Sequence
  Primer
tgccctgactttcaactctgtctccttcctc

SEQ ID NO: 2 - DNA, 35 bp, Artificial Sequence
  Primer
ccagacctaagagcaatcagtgaggaatcagaggc

SEQ ID NO: 3 - DNA, 31 bp, Artificial Sequence
  Primer
gttatctcctaggttggctctgactgtacca

SEQ ID NO: 4 - DNA, 31 bp, Artificial Sequence
  Primer
gtggatgggtagtagtatggaagaaatcggt

SEQ ID NO: 5 - DNA, 31 bp, Artificial Sequence
  Primer
ggtaggacctgatttccttactgcctcttgc

SEQ ID NO: 6 - DNA, 31 bp, Artificial Sequence
  Primer
gataaaagtgaatctgaggcataactgcacc

SEQ ID NO: 7 - DNA, 35 bp, Artificial Sequence
  Primer
tggtggagtatttgatagtgtattaaccttatgtg

SEQ ID NO: 8 - DNA, 36 bp, Artificial Sequence
  Primer
agagaaacctttatctgatatcaaagaatggtcctg

SEQ ID NO: 9 - DNA, 32 bp, Artificial Sequence
  Primer
tgaagtaaaaggtgcactgtaataatccagac

SEQ ID NO: 10 - DNA, 35 bp, Artificial Sequence
  Primer
taatgtcagcttattatattcaatttaaacccacc

SEQ ID NO: 11 - DNA, 22 bp, Artificial Sequence
  Primer
gcacatgacggaggttgtgagg

SEQ ID NO: 12 - DNA, 23 bp, Artificial Sequence
  Primer
gcacatgacggaggttgtgaggc

SEQ ID NO: 13 - DNA, 24 bp, Artificial Sequence
  Primer
gcacatgacggaggttgtgaggcg

SEQ ID NO: 14 - DNA, 21 bp, Artificial Sequence
  Primer
cagcgctcatggtggggggca

SEQ ID NO: 15 - DNA, 22 bp, Artificial Sequence
  Primer
cagcgctcatggtggggggcag

SEQ ID NO: 16 - DNA, 20 bp, Artificial Sequence
  Primer
gtaacagttcctgcatgggc

SEQ ID NO: 17 - DNA, 21 bp, Artificial Sequence
  Primer
gtaacagttcctgcatgggcg

SEQ ID NO: 18 - DNA, 22 bp, Artificial Sequence
  Primer
gtaacagttcctgcatgggcgg

SEQ ID NO: 19 - DNA, 20 bp, Artificial Sequence
  Primer
cctgcatgggcggcatgaac

SEQ ID NO: 20 - DNA, 21 bp, Artificial Sequence
  Primer
cctgcatgggcggcatgaacc

SEQ ID NO: 21 - DNA, 22 bp, Artificial Sequence
  Primer
cctgcatgggcggcatgaaccg

SEQ ID NO: 22 - DNA, 20 bp, Artificial Sequence
  Primer
gtgatgatggtgaggatggg

SEQ ID NO: 23 - DNA, 21 bp, Artificial Sequence
  Primer
gtgatgatggtgaggatgggc

SEQ ID NO: 24 - DNA, 22 bp, Artificial Sequence
  Primer
gtgatgatggtgaggatgggcc

SEQ ID NO: 25 - DNA, 20 bp, Artificial Sequence
  Primer
gacggaacagctttgaggtg

SEQ ID NO: 26 - DNA, 21 bp, Artificial Sequence
  Primer
gacggaacagctttgaggtgc

SEQ ID NO: 27 - DNA, 22 bp, Artificial Sequence
  Primer
gacggaacagctttgaggtgcg

SEQ ID NO: 28 - DNA, 20 bp, Artificial Sequence
  Primer
gtgcctgtcctgggagagac

SEQ ID NO: 29 - DNA, 21 bp, Artificial Sequence
  Primer
gtgcctgtcctgggagagacc

SEQ ID NO: 30 - DNA, 22 bp, Artificial Sequence
  Primer
gtgcctgtcctgggagagaccg

SEQ ID NO: 31 - DNA, 20 bp, Artificial Sequence
  Primer
aacttgtggtagttggagct

SEQ ID NO: 32 - DNA, 21 bp, Artificial Sequence
  Primer
aacttgtggtagttggagctg

SEQ ID NO: 33 - protein, 4 aa, Artificial Sequence
  Linking moiety
GGGG

SEQ ID NO: 34 - DNA, 22 bp, Artificial Sequence
  Primer
aacttgtggtagttggagctgg

SEQ ID NO: 35 - DNA, 20 bp, Artificial Sequence
  Primer
gtcaaggcactcttgcctac

SEQ ID NO: 36 - DNA, 21 bp, Artificial Sequence
  Primer
gtcaaggcactcttgcctacg

SEQ ID NO: 37 - DNA, 22 bp, Artificial Sequence
  Primer
gtcaaggcactcttgcctacgc

SEQ ID NO: 38 - DNA, 20 bp, Artificial Sequence
  Primer
atattctcgacacagcaggt

SEQ ID NO: 39 - DNA, 21 bp, Artificial Sequence
  Primer
atattctcgacacagcaggtc

SEQ ID NO: 40 - DNA, 22 bp, Artificial Sequence
  Primer
atattctcgacacagcaggtca

SEQ ID NO: 41 - protein, 4 aa, Artificial Sequence
  Targeting moiety
CSKC

SEQ ID NO: 42 - DNA, 12 bp, Artificial Sequence
  Primer
ccgcttcctttc

SEQ ID NO: 43 - DNA, 12 bp, Artificial Sequence
  K-RAS antisense
gccaacagctcc

SEQ ID NO: 44 - DNA, 12 bp, Artificial Sequence
  K-RAS mismatch
gccttgtgctcc

SEQ ID NO: 45 - DNA, 12 bp, Artificial Sequence
  cyclin D1 antisense
ctggtgttccat

SEQ ID NO: 46 - DNA, 12 bp, Artificial Sequence
  cyclin D1 mismatch
ctggacaaccat

SEQ ID NO: 47 - DNA, 12 bp, Artificial Sequence
  ERBB2 antisense
catggtgctcac

SEQ ID NO: 48 - DNA, 12 bp, Artificial Sequence
  ERBB2 mismatch
catgcacttcac

SEQ ID NO: 49 - DNA, 12 bp, Artificial Sequence
  c-MYC antisense
gcatcgtcgcgg

SEQ ID NO: 50 - DNA, 12 bp, Artificial Sequence
  c-MYC mismatch
gcatgtctgcgg

SEQ ID NO: 51 - DNA, 12 bp, Artificial Sequence
  p53 antisense
ccccctggctcc

SEQ ID NO: 52 - DNA, 12 bp, Artificial Sequence
  p53 mismatch
cccctaccctcc

SEQ ID NO: 53 - protein, 105 aa, homo sapiens
GPETLCGAELVDALQFVCGDRGFYFNKPTGYGSSSRRAPQTGIVDECCFRSCDLRRLEMYCAPLKPAKSARSVRAQRHTDMPKTQKEVHLKNASRGSAGNKNYRM

SEQ ID NO: 54 - protein, 13 aa, Artificial Sequence
  D-peptide homolog of IGF1
GCSKAPKLPAALC

SEQ ID NO: 55 - protein, 12 aa, Artificial Sequence
  D-peptide homolog of IGF1
CSKAPKLPAAYC